(19) United States
(12) Patent Application Publication
(10) Pub. No.: US 2025/0025451 A1
(43) Pub. Date: Jan. 23, 2025

(54) TREATMENT OF CANCER WITH KDM4 INHIBITORS

(71) Applicants: Celgene Corporation, Summit, NJ (US); Tachyon Therapeutics, Inc., Houston, TX (US)

(72) Inventors: Frank PERABO, Houston, TX (US); Jeffrey A. STAFFORD, Houston, TX (US); Michael CLARKE, Houston, TX (US); Young K. CHEN, San Diego, CA (US)

(21) Appl. No.: 18/554,283

(22) PCT Filed: Apr. 8, 2022
(86) PCT No.: PCT/US2022/024087

§ 371 Date: Oct. 6, 2023

Related U.S. Application Data
(60) Provisional application No. 63/297,960, filed on Jan. 10, 2022.
(60) Provisional application No. 63/252,542, filed on Oct. 5, 2021.
(60) Provisional application No. 63/173,219, filed on Apr. 9, 2021.

(51) Int. Cl. A61K 31/4433; A61P 35/00; G01N 33/68

(52) U.S. Cl. CPC A61K 31/4433; A61P 35/00; G01N 33/6893; G01N 2333/4703; G01N 2800/52

(57) ABSTRACT

Provided herein are compositions and methods for the treatment of a cancer. Said compositions comprise a KDM4 inhibitor.

### CROSS-REFERENCE TO RELATED APPLICATION

This application claims the benefit of U.S. Provisional Patent Application No. 63/173,219, filed on Apr. 9, 2021; U.S. Provisional Patent Application No. 63/252,542, filed on Oct. 5, 2021; and U.S. Provisional Patent Application No. 63/297,960, filed on Jan. 10, 2022, all are hereby incorporated by reference in their entirety.

### BRIEF SUMMARY

Provided herein are compositions and methods for the treatment of cancer. The types of cancer suitable for the methods disclosed herein include, but are not limited to, colorectal cancer, triple negative breast cancer, gastric adenocarcinoma, and diffuse large B-cell non-Hodgkin's lymphoma.

The compositions useful for the methods of treating cancer disclosed herein comprise heterocyclic KDM4 inhibitors described herein.

One embodiment provides a method of treating a cancer in a cancer patient in need thereof, comprising administering to the individual a compound, or pharmaceutically acceptable salt or solvate thereof, having the structure:

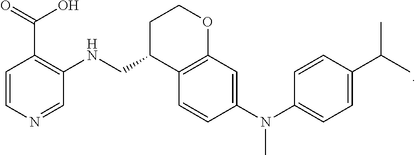 [SMILES: CC(C)c1ccc(N(C)c2ccc3c(c2)OCC[C@H]3CNc2cnccc2C(=O)O)cc1]

Another embodiment provides the method, wherein the cancer patient has been diagnosed with a cancer selected from colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, or T-cell acute lymphoblastic leukemia.

Another embodiment provides a method of reducing tumorigenic cell population in cancer patient in need thereof, comprising administering to the individual a compound, or pharmaceutically acceptable salt or solvate thereof, having the structure:

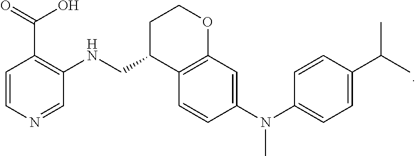 [SMILES: CC(C)c1ccc(N(C)c2ccc3c(c2)OCC[C@H]3CNc2cnccc2C(=O)O)cc1]

Another embodiment provides a method of reducing tumor initiating cell frequency in a cancer patient in need thereof, comprising administering to the individual a compound, or pharmaceutically acceptable salt or solvate thereof, having the structure:

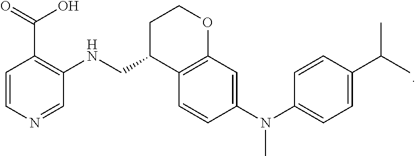 [SMILES: CC(C)c1ccc(N(C)c2ccc3c(c2)OCC[C@H]3CNc2cnccc2C(=O)O)cc1]

Another embodiment provides a method of inhibiting cancer stem cells in a cancer patient in need thereof, comprising administering to the individual a compound, or pharmaceutically acceptable salt or solvate thereof, having the structure:

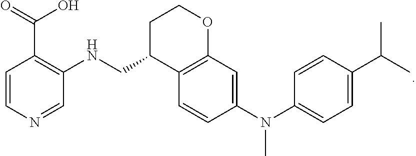 [SMILES: CC(C)c1ccc(N(C)c2ccc3c(c2)OCC[C@H]3CNc2cnccc2C(=O)O)cc1]

### BRIEF DESCRIPTION OF THE DRAWINGS

FIG. 1 illustrates the biochemical activities of Compound 1, a potent PAN KDM4 inhibitor.

FIG. 2 illustrates the reversible and competitive inhibition of H3K9me3 demethylation. Inhibition of H3K3me3 demethylation was measured at various Compound 1 concentrations in the presence of α-KG using time-resolved fluorescence-based (TR-FRET) LANCE® detection.

FIG. 3 illustrates that Compound 1 induces cell cycle arrest in S-phase. Compound 1 increased proportion of cells in S-phase in a dose-dependent manner. Cell cycle analysis was performed with MDA-MB-231 cells and using MultiCycle AV DNA analysis software.

FIG. 4 demonstrates that Compound 1 induces apoptosis in human cancer cell lines.

FIG. 5 demonstrates that Compound 1 exhibits potent anti-proliferative activity across a large panel of cancer cell lines. Evaluation of compound 1 in a panel of PDX/organoid models confirms compound 1 sensitivity in gastric cancers and shows sensitivity to MSI CRC vs. MSS CRC.

FIG. 6 illustrates the dose-dependent inhibition of histone demethylase by Compound 1. Left Panel: Representative immunoblot showing change in staining intensity for actin (red bands) and H3K36me3 (green bands) in response to Compound 1 at various concentrations. Right Panel: Normalized ratio of band intensities for H3K36me3 and actin versus the concentration of Compound 1 (μM). (mean IC50±SD value−0.085±0.004 μM).

FIG. 7 illustrates the reduction of tumorigenic cells upon treatment of tumors with Compound 1. Compound 1 reduced tumor initiating cell (TIC) frequency by 4.4-fold.

FIG. 8 illustrates the in vivo efficacy of Compound 1 in multiple cancer models.

FIG. 9 illustrates inhibition of cell proliferation in the SU60 organoid model.

FIG. 10 In vivo efficacy of Compound 1 in the SU60 patient-derived colorectal cancer xenograft model.

FIG. 11 In vivo efficacy in study no. 1 of Compound 1 in the KYSE-150 human esophageal cancer xenograft model.

FIG. 12 In vivo efficacy in study no. 2 of Compound 1 in the KYSE-150 human esophageal cancer xenograft model.

FIG. 13 In vivo efficacy of Compound 1 in the COH70 patient-derived triple negative breast cancer xenograft model.

FIG. 14 In vivo Efficacy of Compound 1 in the GXA-3036 gastric adenocarcinoma patient-derived xenograft model.

FIG. 15 In vivo Efficacy of Compound 1 in the OCI-LY19 human diffuse large B-cell non-Hodgkin's lymphoma xenograft model.

FIG. 16 illustrates flow cytometry analysis of tumorigenic cell population after Compound 1 treatment.

FIG. 17 illustrates the results of the in vivo tumorigenicity functional assay after Compound 1 treatment.

FIG. 18 illustrates a heatmap of the mutation status of genes in various pathways in different colorectal cancer cell lines. The respective Microsatellite instability (MSI-H), CpG island methylator phenotype (CMIP), and MLH1 methylation status as well as the sensitivity of these cell lines are to Compound 1 are also shown.

FIG. 19 illustrates a heatmap showing the MSI-H status and respective MMRpath gene mutations of various colorectal cancer cell lines studied.

FIG. 20 illustrates that genes PNUTS (PPP1R10), ANK1, IBA57, SOWAHD, MF12-AS1, and CECR1 exhibited dose-dependent changes following treatment with Compound 1 in vivo.

FIG. 21 illustrates that ChIP-seq analysis in MDA-MB-231 TNBC cells shows KDM4 occupancy at the PNUTS (PPP1R10) promoter.

FIG. 22 illustrates the heatmap representation of single cells gene expression analysis (RT-qPCR) from SU60 xenograft tumors treated with vehicle control or Compound 1. A) Heatmap representation of single cells gene expression analysis (RT-qPCR) from SU60 xenograft tumors treated with vehicle control or Compound 1. Gene expression levels are colored-coded with red for high expression, green low expression and gray not expressed. Three clusters of cells were identified based on shared patterns of gene expression using a gene-set of immature and mature cell markers. On the far right of the heatmap a red/blue ribbon displays the distribution of 335 vehicle-treated (blue) and 332 compound 1-treated (red) single cells. The top dendrogram clusters genes with similar expression pattern across all cells. Gene expression (in Ct values) was mean-centered at 0 and divided by 2.5 time the standard deviation. B) This graph represents the total number of vehicle-treated and compound 1 treated cells within each cluster. P-values (Fisher's exact test) are represented on top of the bars.

### INCORPORATION BY REFERENCE

All publications, patents, and patent applications mentioned in this specification are herein incorporated by reference for the specific purposes identified herein.

### DETAILED DESCRIPTION

### Certain Terminology

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Application s of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

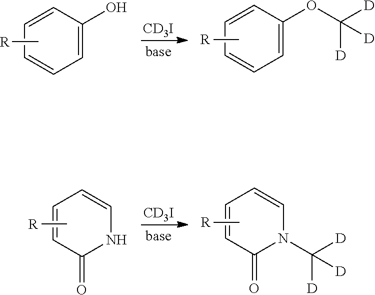

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

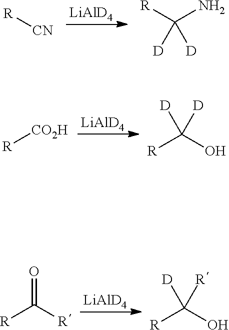

Deuterium gas and palladium catalyst are employed to reduce unsaturated carbon-carbon linkages and to perform a reductive substitution of aryl carbon-halogen bonds as illustrated, by way of example only, in the reaction schemes below.

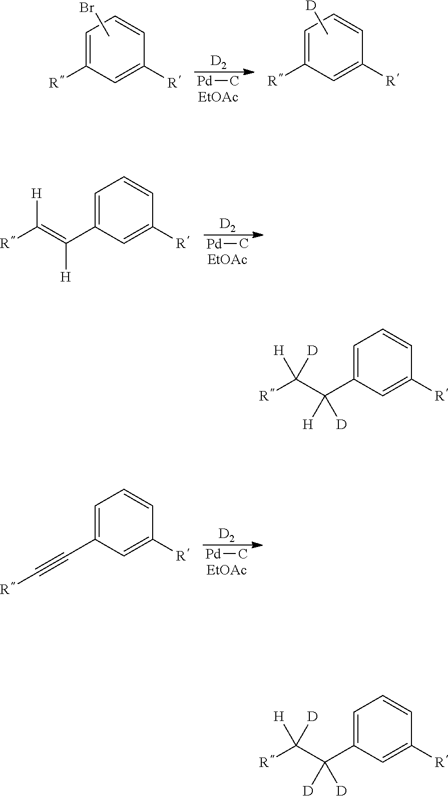

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of heterocyclic KDM4 inhibitor described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or“palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made. The term “treating”, as used herein, unless otherwise indicated, means reversing, alleviating, inhibiting the progress of, or preventing the disorder or condition to which such term applies, or one or more symptoms of such disorder or condition. In some embodiments, the tern “treating” includes slowing or delaying the progression of the disease or disorder to which the term is applied. Additionally, in some embodiments, the term “treating” is applied to one or more of the complications resulting from the disease or disorder to which the term is applied. The term “treatment”, as used herein, unless otherwise indicated, refers to the act of treating as “treating” is defined immediately above.

The term “tumor,” or “cancer” as used herein, and unless otherwise specified, refers to a neoplastic cell growth, and includes pre-cancerous and cancerous cells and tissues. Tumors usually present as a lesion or lump. As used herein, “treating” a tumor means that one or more symptoms of the disease, such as the tumor itself, vascularization of the tumor, or other parameters by which the disease is characterized, are reduced, ameliorated, inhibited, placed in a state of remission, or maintained in a state of remission. “Treating” a tumor also means that one or more hallmarks of the tumor may be eliminated, reduced or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries.

The term “refractory” or “refractory to therapy” indicates that the patients have never responded to therapy.

The term “relapsed” or “relapsed after therapy” indicates that patients, after initially responding to therapy, have progressive disease due to acquired resistance and/or intolerance.

The term “resistance to therapy” or “acquired resistance to therapy” indicates the patients, after initially responding to therapy, have progressive disease due to clinical or molecular resistance to the therapy. The acquired resistance can result from emergence of resistant mutations in the molecular target of the therapy, or in the development of physiological functions such as efflux pumps.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The term “tumorigenic” indicates a cell that is capable of forming or tending to form tumors.

The term “tumorigenic cell population” indicates a population of cells that have neoplastic properties and are capable of forming tumors.

The term “tumor initiating cell” or “cancer stem cell” refers to cell(s) that produce the various differentiated progeny that make up the bulk of the rapidly dividing tumor.

Other aspects, advantages, and features of the invention will become apparent from the detailed description below.

### Histone Demethylase

Chromatin is the complex of DNA and protein that makes up chromosomes. Histones are the major protein component of chromatin, acting as spools around which DNA winds. Changes in chromatin structure are affected by covalent modifications of histone proteins and by non-histone binding proteins. Several classes of enzymes are known which can covalently modify histones at various sites.

Proteins can be post-translationally modified by methylation on amino groups of lysines and guanidino groups of arginines or carboxymethylated on aspartate, glutamate, or on the C-terminus of the protein. Post-translational protein methylation has been implicated in a variety of cellular processes such as RNA processing, receptor mediated signaling, and cellular differentiation. Post-translational protein methylation is widely known to occur on histones, such reactions known to be catalyzed by histone methyltransferases, which transfer methyl groups from S-adenyosyl methionine (SAM) to histones. Histone methylation is known to participate in a diverse range of biological processes including heterochromatin formation, X-chromosome inactivation, and transcriptional regulation (Lachner et al., (2003) J. Cell Sci. 116:2117-2124; Margueron et al., (2005) Curr. Opin. Genet. Dev. 15:163-176).

Unlike acetylation, which generally correlates with transcriptional activation, whether histone methylation leads to transcription activation or repression depends on the particular site of methylation and the degree of methylation (e.g., whether a particular histone lysine residue is mono-, di-, or tri-methylated). However, generally, methylation on H3K9, H3K27 and H4K20 is linked to gene silencing, while methylation on H3K4, H3K36, and H3K79 is generally associated with active gene expression. In addition, tri- and di-methylation of H3K4 generally marks the transcriptional start sites of actively transcribed genes, whereas mono-methylation of H3K4 is associated with enhancer sequences.

A “demethylase” or “protein demethylase,” as referred to herein, refers to an enzyme that removes at least one methyl group from an amino acid side chain. Some demethylases act on histones, e.g., act as a histone H3 or H4 demethylase. For example, an H3 demethylase may demethylate one or more of H3K4, H3K9, H3K27, H3K36 and/or H3K79. Alternately, an H4 demethylase may demethylate histone H4K20. Demethylases are known which can demethylate either a mono-, di- and/or a tri-methylated substrate. Further, histone demethylases can act on a methylated core histone substrate, a mononucleosome substrate, a dinucleosome substrate and/or an oligonucleosome substrate, peptide substrate and/or chromatin (e.g., in a cell-based assay).

The first lysine demethylase discovered was lysine specific demethylase 1 (LSD1/KDM1), which demethylates both mono- and di-methylated H3K4 or H3K9, using flavin as a cofactor. A second class of Jumonji C (JmjC) domain containing histone demethylases were predicted, and confirmed when a H3K36 demethylase was found using a formaldehyde release assay, which was named JmjC domain containing histone demethylase 1 (JHDM1/KDM2A).

More JmjC domain-containing proteins were subsequently identified and they can be phylogenetically clustered into seven subfamilies: JHDM1, JHDM2, JHDM3, JMJD2, JARID, PHF2/PHF8, UTX/UTY, and JmjC domain only.

### JMJD2 Family

The JMJD2 family of proteins are a family of histone-demethylases known to demethylate tri- and di-methylated H3-K9, and were the first identified histone tri-methyl demethylases. In particular, ectopic expression of JMJD2 family members was found to dramatically decrease levels of tri-and di-methylated H3-K9, while increasing levels of mono-methylated H3-K9, which delocalized Heterochromatin Protein 1 (HP1) and reduced overall levels of heterochromatin in vivo. Members of the JMJD2 subfamily of jumonji proteins include JMJD2C and its homologues JMJD2A, JMJD2B, JMJD2D and JMJD2E. Common structural features found in the JMJD2 subfamily of Jumonji proteins include the JmjN, JmjC, PHD and Tdr sequences.

JMJD2C, also known as GASC1 and KDM4C, is known to demethylate tri-methylated H3K9 and H3K36. Histone demethylation by JMJD2C occurs via a hydroxylation reaction dependent on iron and α-ketoglutarate, wherein oxidative decarboxylation of α-ketoglutarate by JMJD2C produces carbon dioxide, succinate, and ferryl and ferryl subsequently hydroxylates a methyl group of lysine H3K9, releasing formaldehyde. JMJD2C is known to modulate regulation of adipogenesis by the nuclear receptor PPARγ and is known to be involved in regulation of self-renewal in embryonic stem cells.

KDM4 histone lysine demethylase is an epigenetic regulator and key oncogenic driver across multiple tumor types. The KDM4 family consists of four main isoforms (KDM4A, B, C, D); all have been implicated in epigenetic dysregulation in various cancers (Zack et al., 2013). KDM4 controls transition between transcriptionally silent and active chromatin states via removal of methyl marks on histone H3K9 and histone H3K36. KDM4 is also necessary for self-renewal of embryonic stem cells and the generation of induced pluripotent stem cells (Das et al. 2014; Kim et al. 2010; Loh et al. 2007; Wang et al. 2010). Overexpression of KDM4 is linked to more aggressive disease and poorer clinical outcomes (Jia et al. 2020; Bur et al. 2016; Soini et al. 2015). Functional redundancy and cross-activity have been observed across KDM4 isoforms; selective inhibition of one isoform appears to not be effective (Agger et al. 2016; Pedersen et al. 2016).

### Heterocyclic KDM4 Inhibitor

The heterocyclic KDM4 inhibitor described herein refers to Compound 1 having the structure below, and the chemical name 3-({[(4R)-7-{methyl[4-(propan-2-yl)phenyl]amino}-3,4-dihydro-2H-1-benzopyran-4-yl]methyl}amino)pyridine-4-carboxylic acid:

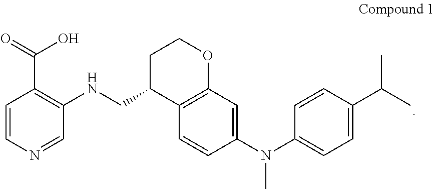 [SMILES: CC(C)c1ccc(N(C)c2ccc3c(c2)OCC[C@H]3CNc2cnccc2C(=O)O)cc1]

Compound 1 has been previously disclosed in PCT patent publication WO2015/200709 and related patent applications and granted patents, such as U.S. Pat. No. 9,242,968, which are incorporated by reference in their entirety. Compound 1 is a pan inhibitor of KDM4 that simultaneously targets multiple isoforms of KDM4. Throughout this disclosure when reference is made to a heterocyclic KDM4 inhibitor, or pharmaceutically acceptable salts or solvates thereof, the reference is to Compound 1.

### Cancer and Methods of Treatment

In one embodiment described herein is a method for inhibiting a histone-demethylase enzyme comprising contacting the enzyme with Compound 1 as disclosed herein, wherein the histone-demethylase enzyme comprises KDM4. In certain aspects, disclosed herein is a method of treating a cancer in an individual in need thereof, comprising administering an effective amount of a heterocyclic KDM4 inhibitor described herein to the individual. In certain aspects, disclosed herein is a heterocyclic KDM4 inhibitor described herein for use in treating a cancer. In certain aspects, disclosed herein is a heterocyclic KDM4 inhibitor described herein for use in preparation of a medicament for treating a cancer.

One embodiment provides a method of treating a cancer in a cancer patient in need thereof, comprising administering to the individual a compound, or pharmaceutically acceptable salt or solvate thereof, having the structure:

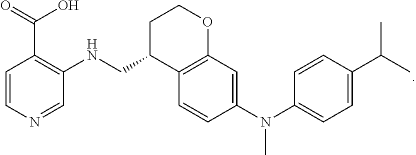 [SMILES: CC(C)c1ccc(N(C)c2ccc3c(c2)OCC[C@H]3CNc2cnccc2C(=O)O)cc1]

Another embodiment provides the method, wherein the cancer patient has been diagnosed with a cancer selected from colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, or T-cell acute lymphoblastic leukemia.

Another embodiment provides the method, wherein the cancer patient has been diagnosed with colorectal cancer. Another embodiment provides the method, wherein the cancer patient has been diagnosed with esophageal cancer. Another embodiment provides the method, wherein the cancer patient has been diagnosed with triple negative breast cancer. Another embodiment provides the method, wherein the cancer patient has been diagnosed with gastric cancer. Another embodiment provides the method, wherein the cancer patient has been diagnosed with lymphoma. Another embodiment provides the method, wherein the cancer patient has been diagnosed with gastric adenocarcinoma. Another embodiment provides the method, wherein the cancer patient has been diagnosed with diffuse large B-cell non-Hodgkin's lymphoma. Another embodiment provides the method, wherein the cancer patient has been diagnosed with acute T-cell leukemia. Another embodiment provides the method, wherein the cancer patient has been diagnosed with a cancer selected from esophageal squamous cell carcinoma. Another embodiment provides the method, wherein the cancer patient has been diagnosed with multiple myeloma. Another embodiment provides the method, wherein the cancer patient has been diagnosed with acute myeloid leukemia. Another embodiment provides the method, wherein the cancer patient has been diagnosed with colorectal adenocarcinoma. Another embodiment provides the method, wherein the cancer patient has been diagnosed with colorectal carcinoma. Another embodiment provides the method, wherein the cancer patient has been diagnosed with pancreatic cancer. Another embodiment provides the method, wherein the cancer patient has been diagnosed with pancreatic carcinoma. Another embodiment provides the method, wherein the cancer patient has been diagnosed with breast carcinoma. Another embodiment provides the method, wherein the cancer patient has been diagnosed with T-cell acute lymphoblastic leukemia. Another embodiment provides the method, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma. Another embodiment provides the method, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia.

Another embodiment provides the method, wherein the cancer patient has been diagnosed with a cancer selected from intestinal cancer, intestinal adenocarcinoma, squamous cell carcinoma of the upper digestive tract. Another embodiment provides the method, wherein the cancer patient has been diagnosed with a cancer selected from stomach cancer, stomach signet ring adenocarcinoma, adenocarcinoma of the stomach, or adenosquamous carcinoma. Another embodiment provides the method, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

Another embodiment provides the method, wherein the cancer patient has been diagnosed with skin cancer. Another embodiment provides the method, wherein the cancer patient has been diagnosed with a cancer selected from thyroid cancer, or thyroid follicular carcinoma. Another embodiment provides the method, wherein the cancer patient has been diagnosed with a cancer selected from breast cancer, or breast ductal carcinoma. Another embodiment provides the method, wherein the cancer patient has been diagnosed with a cancer selected from liver cancer, or hepatocellular carcinoma. Another embodiment provides the method, wherein the cancer patient has been diagnosed with a cancer selected from a CNS cancer, astrocytoma grade IV, or gliosarcoma. Another embodiment provides the method, wherein the cancer patient has been diagnosed with bone cancer. Another embodiment provides the method, wherein the cancer patient has been diagnosed with a cancer selected from kidney cancer, clear cell renal cell carcinoma, renal cell carcinoma, urinary tract cancer, or urinary tract transitional cell carcinoma. Another embodiment provides the method, wherein the cancer is relapsed after prior therapy, refractory to prior therapy, or acquired resistance to prior therapy.

Another embodiment provides a method of reducing tumorigenic cell population in cancer patient in need thereof, comprising administering to the individual a compound, or pharmaceutically acceptable salt or solvate thereof, having the structure:

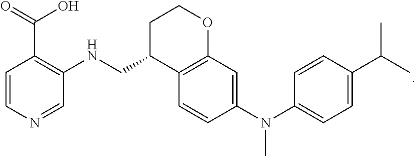 [SMILES: CC(C)c1ccc(N(C)c2ccc3c(c2)OCC[C@H]3CNc2cnccc2C(=O)O)cc1]

Another embodiment provides the method, wherein the population of tumorigenic cell is reduced from about 1.5-fold to about 100-fold. Another embodiment provides the method, wherein the population of tumorigenic cell is reduced about 1.5-fold, about 2.0-fold, about 2.5-fold, about 3.0-fold, about 3.5-fold, about 4.0-fold, about 4.5-fold, about 5.0-fold, about 6.0-fold, about 7.0-fold, about 8.0-fold, about 9.0-fold, about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, or about 100-fold. Another embodiment provides the method, wherein the population of tumorigenic cell is reduced about 1.5-fold, about 2.0-fold, about 2.5-fold, about 3.0-fold, about 3.5-fold, about 4.0-fold, about 4.5-fold, or about 5.0-fold. Another embodiment provides the method, wherein the cancer patient has been diagnosed with a cancer selected from colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, or T-cell acute lymphoblastic leukemia. Another embodiment provides the method, wherein the cancer patient has been diagnosed with colorectal cancer. Another embodiment provides the method, wherein the cancer patient has been diagnosed with esophageal cancer. Another embodiment provides the method, wherein the cancer patient has been diagnosed with triple negative breast cancer. Another embodiment provides the method, wherein the cancer patient has been diagnosed with gastric cancer. Another embodiment provides the method, wherein the cancer patient has been diagnosed with lymphoma. Another embodiment provides the method, wherein the cancer patient has been diagnosed with gastric adenocarcinoma. Another embodiment provides the method, wherein the cancer patient has been diagnosed with diffuse large B-cell non-Hodgkin's lymphoma. Another embodiment provides the method, wherein the cancer patient has been diagnosed with acute T-cell leukemia. Another embodiment provides the method, wherein the cancer patient has been diagnosed with esophageal squamous cell carcinoma. Another embodiment provides the method, wherein the cancer patient has been diagnosed with multiple myeloma. Another embodiment provides the method, wherein the cancer patient has been diagnosed with acute myeloid leukemia. Another embodiment provides the method, wherein the cancer patient has been diagnosed with colorectal adenocarcinoma. Another embodiment provides the method, wherein the cancer patient has been diagnosed with colorectal carcinoma. Another embodiment provides the method, wherein the cancer patient has been diagnosed with pancreatic cancer. Another embodiment provides the method, wherein the cancer patient has been diagnosed with pancreatic carcinoma. Another embodiment provides the method, wherein the cancer patient has been diagnosed with breast carcinoma. Another embodiment provides the method, wherein the cancer patient has been diagnosed with T-cell acute lymphoblastic leukemia. Another embodiment provides the method, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma. Another embodiment provides the method, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia. Another embodiment provides the method, wherein the cancer patient has been diagnosed with a cancer selected from intestinal cancer, intestinal adenocarcinoma, squamous cell carcinoma of the upper digestive tract. Another embodiment provides the method, wherein the cancer patient has been diagnosed with a cancer selected from stomach cancer, stomach signet ring adenocarcinoma, adenocarcinoma of the stomach, or adenosquamous carcinoma. Another embodiment provides the method, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

Another embodiment provides the method, wherein the cancer patient has been diagnosed with skin cancer. Another embodiment provides the method, wherein the cancer patient has been diagnosed with a cancer selected from thyroid cancer, or thyroid follicular carcinoma. Another embodiment provides the method, wherein the cancer patient has been diagnosed with a cancer selected from breast cancer, or breast ductal carcinoma. Another embodiment provides the method, wherein the cancer patient has been diagnosed with a cancer selected from liver cancer, or hepatocellular carcinoma. Another embodiment provides the method, wherein the cancer patient has been diagnosed with a cancer selected from a CNS cancer, astrocytoma grade IV, or gliosarcoma. Another embodiment provides the method, wherein the cancer patient has been diagnosed with bone cancer. Another embodiment provides the method, wherein the cancer patient has been diagnosed with a cancer selected from kidney cancer, clear cell renal cell carcinoma, renal cell carcinoma, urinary tract cancer, or urinary tract transitional cell carcinoma. Another embodiment provides the method, wherein the cancer is relapsed after prior therapy, refractory to prior therapy, or acquired resistance to prior therapy.

Another embodiment provides a method of reducing tumor initiating cell frequency in a cancer patient in need thereof, comprising administering to the individual a compound, or pharmaceutically acceptable salt or solvate thereof, having the structure:

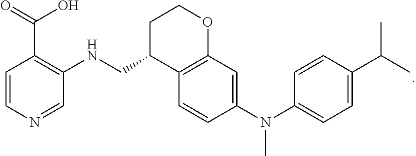 [SMILES: CC(C)c1ccc(N(C)c2ccc3c(c2)OCC[C@H]3CNc2cnccc2C(=O)O)cc1]

Another embodiment provides the method, wherein the tumor initiating cell frequency is reduced from about 1.5-fold to about 100-fold. Another embodiment provides the method, wherein the tumor initiating cell frequency is reduced about 1.5-fold, about 2.0-fold, about 2.5-fold, about 3.0-fold, about 3.5-fold, about 4.0-fold, about 4.5-fold, about 5.0-fold, about 6.0-fold, about 7.0-fold, about 8.0-fold, about 9.0-fold, or about 10-fold. Another embodiment provides the method, wherein the tumor initiating cell frequency is reduced to a frequency of about 1-in-100 cells to about 1-in-10,000 cells. Another embodiment provides the method, wherein the tumor initiating cell frequency is reduced to a frequency of about 1-in-100 cells, about 1-in-200 cells, about 1-in-300 cells, about 1-in-400 cells, about 1-in-500 cells, about 1-in-600 cells, about 1-in-700 cells, about 1-in-800 cells, about 1-in-900 cells, about 1-in-1,000 cells, about 1-in-1,200 cells, about 1-in-1,400 cells, about 1-in-1,600 cells, about 1-in-1,800 cells, about 1-in-2,000 cells, about 1-in-3,000 cells, about 1-in-4,000 cells, about 1-in-5,000 cells, about 1-in-6,000 cells, about 1-in-7,000 cells, about 1-in-8,000 cells, about 1-in-9,000 cells, or to about 1-in-10,000 cells. Another embodiment provides the method, wherein the tumor initiating cell frequency is reduced to a frequency of about 1-in-200 cells, about 1-in-300 cells, about 1-in-400 cells, about 1-in-500 cells, about 1-in-600 cells, about 1-in-700 cells, about 1-in-800 cells, about 1-in-900 cells, about 1-in-1,000 cells, or about 1-in-1,200 cells. Another embodiment provides the method, wherein the cancer patient has been diagnosed with a cancer selected from colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, or T-cell acute lymphoblastic leukemia. Another embodiment provides the method, wherein the cancer patient has been diagnosed with colorectal cancer. Another embodiment provides the method, wherein the cancer patient has been diagnosed with esophageal cancer. Another embodiment provides the method, wherein the cancer patient has been diagnosed with triple negative breast cancer. Another embodiment provides the method, wherein the cancer patient has been diagnosed with gastric cancer. Another embodiment provides the method, wherein the cancer patient has been diagnosed with lymphoma. Another embodiment provides the method, wherein the cancer patient has been diagnosed with a cancer selected from gastric adenocarcinoma. Another embodiment provides the method, wherein the cancer patient has been diagnosed with diffuse large B-cell non-Hodgkin's lymphoma. Another embodiment provides the method, wherein the cancer patient has been diagnosed with acute T-cell leukemia. Another embodiment provides the method, wherein the cancer patient has been diagnosed with esophageal squamous cell carcinoma. Another embodiment provides the method, wherein the cancer patient has been diagnosed with multiple myeloma. Another embodiment provides the method, wherein the cancer patient has been diagnosed with acute myeloid leukemia. Another embodiment provides the method, wherein the cancer patient has been diagnosed with colorectal adenocarcinoma. Another embodiment provides the method, wherein the cancer patient has been diagnosed with colorectal carcinoma. Another embodiment provides the method, wherein the cancer patient has been diagnosed with pancreatic cancer. Another embodiment provides the method, wherein the cancer patient has been diagnosed with pancreatic carcinoma. Another embodiment provides the method, wherein the cancer patient has been diagnosed with breast carcinoma. Another embodiment provides the method, wherein the cancer patient has been diagnosed with T-cell acute lymphoblastic leukemia. Another embodiment provides the method, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma. Another embodiment provides the method, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia. Another embodiment provides the method, wherein the cancer patient has been diagnosed with a cancer selected from intestinal cancer, intestinal adenocarcinoma, squamous cell carcinoma of the upper digestive tract. Another embodiment provides the method, wherein the cancer patient has been diagnosed with a cancer selected from stomach cancer, stomach signet ring adenocarcinoma, adenocarcinoma of the stomach, or adenosquamous carcinoma. Another embodiment provides the method, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

Another embodiment provides the method, wherein the cancer patient has been diagnosed with skin cancer. Another embodiment provides the method, wherein the cancer patient has been diagnosed with a cancer selected from thyroid cancer, or thyroid follicular carcinoma. Another embodiment provides the method, wherein the cancer patient has been diagnosed with a cancer selected from breast cancer, or breast ductal carcinoma. Another embodiment provides the method, wherein the cancer patient has been diagnosed with a cancer selected from liver cancer, or hepatocellular carcinoma. Another embodiment provides the method, wherein the cancer patient has been diagnosed with a cancer selected from a CNS cancer, astrocytoma grade IV, or gliosarcoma. Another embodiment provides the method, wherein the cancer patient has been diagnosed with bone cancer. Another embodiment provides the method, wherein the cancer patient has been diagnosed with a cancer selected from kidney cancer, clear cell renal cell carcinoma, renal cell carcinoma, urinary tract cancer, or urinary tract transitional cell carcinoma. Another embodiment provides the method, wherein the cancer is relapsed after prior therapy, refractory to prior therapy, or acquired resistance to prior therapy.

Another embodiment provides a method of inhibiting cancer stem cells in a cancer patient in need thereof, comprising administering to the individual a compound, or pharmaceutically acceptable salt or solvate thereof, having the structure:

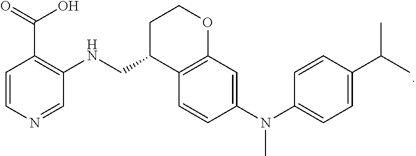 [SMILES: CC(C)c1ccc(N(C)c2ccc3c(c2)OCC[C@H]3CNc2cnccc2C(=O)O)cc1]

Another embodiment provides the method, wherein the cancer patient has been diagnosed with a cancer selected from colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, or T-cell acute lymphoblastic leukemia. Another embodiment provides the method, wherein the cancer patient has been diagnosed with colorectal cancer. Another embodiment provides the method, wherein the cancer patient has been diagnosed with esophageal cancer. Another embodiment provides the method, wherein the cancer patient has been diagnosed with triple negative breast cancer. Another embodiment provides the method, wherein the cancer patient has been diagnosed with gastric cancer. Another embodiment provides the method, wherein the cancer patient has been diagnosed with lymphoma. Another embodiment provides the method, wherein the cancer patient has been diagnosed with gastric adenocarcinoma. Another embodiment provides the method, wherein the cancer patient has been diagnosed with diffuse large B-cell non-Hodgkin's lymphoma. Another embodiment provides the method, wherein the cancer patient has been diagnosed with acute T-cell leukemia. Another embodiment provides the method, wherein the cancer patient has been diagnosed with esophageal squamous cell carcinoma. Another embodiment provides the method, wherein the cancer patient has been diagnosed with multiple myeloma. Another embodiment provides the method, wherein the cancer patient has been diagnosed with acute myeloid leukemia. Another embodiment provides the method, wherein the cancer patient has been diagnosed with colorectal adenocarcinoma. Another embodiment provides the method, wherein the cancer patient has been diagnosed with colorectal carcinoma. Another embodiment provides the method, wherein the cancer patient has been diagnosed with pancreatic cancer. Another embodiment provides the method, wherein the cancer patient has been diagnosed with pancreatic carcinoma. Another embodiment provides the method, wherein the cancer patient has been diagnosed with breast carcinoma. Another embodiment provides the method, wherein the cancer patient has been diagnosed with T-cell acute lymphoblastic leukemia. Another embodiment provides the method, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma. Another embodiment provides the method, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia.
Another embodiment provides the method, wherein the cancer patient has been diagnosed with a cancer selected from intestinal cancer, intestinal adenocarcinoma, squamous cell carcinoma of the upper digestive tract. Another embodiment provides the method, wherein the cancer patient has been diagnosed with a cancer selected from stomach cancer, stomach signet ring adenocarcinoma, adenocarcinoma of the stomach, or adenosquamous carcinoma. Another embodiment provides the method, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

Another embodiment provides the method, wherein the cancer patient has been diagnosed with skin cancer. Another embodiment provides the method, wherein the cancer patient has been diagnosed with a cancer selected from thyroid cancer, or thyroid follicular carcinoma. Another embodiment provides the method, wherein the cancer patient has been diagnosed with a cancer selected from breast cancer, or breast ductal carcinoma. Another embodiment provides the method, wherein the cancer patient has been diagnosed with a cancer selected from liver cancer, or hepatocellular carcinoma. Another embodiment provides the method, wherein the cancer patient has been diagnosed with a cancer selected from a CNS cancer, astrocytoma grade IV, or gliosarcoma. Another embodiment provides the method, wherein the cancer patient has been diagnosed with bone cancer. Another embodiment provides the method, wherein the cancer patient has been diagnosed with a cancer selected from kidney cancer, clear cell renal cell carcinoma, renal cell carcinoma, urinary tract cancer, or urinary tract transitional cell carcinoma. Another embodiment provides the method, wherein the cancer is relapsed after prior therapy, refractory to prior therapy, or acquired resistance to prior therapy.

### Microsatellite Instability (MSI-H) Status

Microsatellite (MS), also called Short Tandem Repeats (STRs) or Simple Sequence Repeat (SSRs), consists of repeated sequences of 1-6 nucleotides (see, e.g., Garrido-Ramos (2017) Genes 8(9):230). The distribution characteristics of MS are different from 15 to 65 nucleotides tandem repeats of small satellite DNA. Depending on the frequency of MSI, it can be distinguished into three types: high microsatellite instability (MSI-H), low microsatellite instability (MSI-L) and microsatellite stability (MSS) (see, e.g., Li, et al., (2020) Cancer Cell International 20:16). Due to deficiency of mismatch repair (MMR) genes in tumor cells or defects in the process of replication repair, the patients with high microsatellite instability (MSI-H) tumors may show higher sensitivity to treatments and benefit from immunotherapy.

In certain embodiments of the methods provided herein, the method further comprises determining high microsatellite instability (MSI-H) status of the cancer patient. In certain embodiments of the methods provided herein, the method further comprises determining whether the cancer patient is diagnosed with high microsatellite instability (MSI-H).

In certain embodiments of the methods provided herein, the cancer patient has been determined to have a high microsatellite instability (MSI-H) status. In certain embodiments of the methods provided herein, the cancer patient has been diagnosed with a cancer of high microsatellite instability (MSI-H).

Another embodiment provides a method of treating a cancer in a cancer patient in need thereof, comprising (a) determining high microsatellite instability (MSI-H) status of the cancer patient, and (b) administering therapeutically effective amount of Compound 1, or a pharmaceutically acceptable salt or a solvate thereof to the cancer patient if the cancer patient is determined to be a MSI-H cancer patient. In some embodiments, the cancer patient has been diagnosed with colorectal cancer. In some embodiments, the cancer patient has been diagnosed with colorectal cancer with MSI-H status.

Another embodiment provides a method of treating a cancer having a high microsatellite instability (MSI-H) status in a cancer patient in need thereof, comprising administering therapeutically effective amount of Compound 1, or a pharmaceutically acceptable salt or a solvate thereof to a patient that is determined to be a MSI-H cancer patient. In some embodiments, the cancer patient has been diagnosed with colorectal cancer.

Another embodiment provides a method of treating a high microsatellite instability (MSI-H) cancer patient, comprising administering therapeutically effective amount of Compound 1, or a pharmaceutically acceptable salt or a solvate thereof to a patient that is determined to be a MSI-H cancer patient. In some embodiments, the cancer patient has been diagnosed with colorectal cancer.

Another embodiment provides a method of reducing tumorigenic cell population in cancer patient in need thereof, comprising (a) determining high microsatellite instability (MSI-H) status of the cancer patient, and (b) administering therapeutically effective amount of Compound 1, or a pharmaceutically acceptable salt or a solvate thereof to a patient that is determined to be a MSI-H cancer patient. In some embodiments, the cancer patient has been diagnosed with colorectal cancer. In some embodiments, the cancer patient has been diagnosed with colorectal cancer with MSI-H status.

Another embodiment provides a method of reducing tumor initiating cell frequency in a cancer patient in need thereof, comprising (a) determining high microsatellite instability (MSI-H) status of the cancer patient, and (b) administering therapeutically effective amount of Compound 1, or a pharmaceutically acceptable salt or a solvate thereof to a patient that is determined to be a MSI-H cancer patient. In some embodiments, the cancer patient has been diagnosed with colorectal cancer. In some embodiments, the cancer patient has been diagnosed with colorectal cancer with MSI-H status.

Another embodiment provides a method of inhibiting cancer stem cells in a cancer patient in need thereof, comprising (a) determining high microsatellite instability (MSI-H) status of the cancer patient, and (b) administering therapeutically effective amount of Compound 1, or a pharmaceutically acceptable salt or a solvate thereof to a patient that is determined to be a MSI-H cancer patient. In some embodiments, the cancer patient has been diagnosed with colorectal cancer. In some embodiments, the cancer patient has been diagnosed with colorectal cancer with MSI-H status.

Microsatellite instability (MSI-H) status can be detected or determined using methods known to those skilled in the art, including but not limited to Next-Generation Sequencing (NGS), fluorescent multiplex Polymerase Chain Reaction (PCR), capillary electrophoresis (CE), immunohistochemistry (IHC), single-molecule molecular inversion probes (smMIPs), and MSI calculation (e.g., MANTIS). In one embodiment, MSI-H status is determined by Next-Generation Sequencing (NGS). In another embodiment, MSI-H status is determined by fluorescent multiplex PCR. In yet another embodiment, MSI-H status is determined by capillary electrophoresis (CE). In yet another embodiment, MSI-H status is determined by immunohistochemistry (IHC). In yet another embodiment, MSI-H status is determined by single-molecule molecular inversion probes (smMIPs). In yet another embodiment, MSI-H status is determined by MSI calculation.

In certain embodiments, the MSI-H has two or more repeat loci microsatellite markers selected from the group consisting of BAT-25, BAT-26, D2S123, D5S346 and D17S250. In certain embodiments, the cancer patient is determined to have a MSI-H status when the cancer patient is determined to have two or more repeat loci microsatellite markers selected from the group consisting of BAT-25, BAT-26, D2S123, D5 S346 and D17S250.

### Biomarkers and Methods of Use Thereof

The methods provided herein are based, in part, on the finding that detectable increase or decrease in certain biomarkers upon compound treatment are observed in cell culture and organoids of cancer (e.g., colorectal cancer), responsive to a given treatment, e.g., Compound 1 having a structure of:

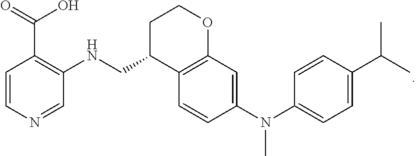 [SMILES: CC(C)c1ccc(N(C)c2ccc3c(c2)OCC[C@H]3CNc2cnccc2C(=O)O)cc1]

or a pharmaceutically acceptable salt or solvate thereof, and that the levels of these biomarkers may be used for predicting the responsiveness of the patients to the treatment.

A “biological marker” or “biomarker” is a substance, the change and/or the detection of which indicates a particular biological state. In some embodiments, the indication is the responsiveness of a disease, e.g., cancer (e.g., colorectal cancer), to a given treatment (e.g., Compound 1 or a pharmaceutically acceptable salt or solvate thereof).

As described in the Examples and shown in the figures, the levels of certain proteins and/or mRNAs change in response to Compound 1. These biomarkers include PNUTS (PPP1R10), ANK1, I1BA57, SOWAHD, MF12-AS1, and CECR1. Each of the biomarkers provided herein includes various isoforms, phosphorylated forms, cleaved forms, modified forms, and splicing variants thereof. In some embodiments, the levels of the isoforms, phosphorylated forms, cleaved forms, modified forms, and/or splicing variants of these biomarkers increase or decrease in response to the compound treatment, and thus these isoforms, phosphorylated forms, cleaved forms, modified forms, and/or splicing variants of the biomarkers can be used to predict a patient's response.

In one aspect, provided herein are methods of identifying a cancer patient with a cancer likely to be responsive to a treatment compound, comprising: (a) administering the treatment compound to the patient; (b) obtaining a sample from the patient; (c) determining the level of a biomarker in the sample; and (d) diagnosing the patient as being likely to be responsive to the treatment compound if the level of the biomarker in the sample is different from a reference level of the biomarker; wherein the treatment compound is Compound 1, or a pharmaceutically acceptable salt or a solvate thereof. In one embodiment, the biomarker is PNUTS.

In another aspect, provided herein are methods of identifying a cancer patient having cancer who is likely to be responsive to a treatment compound, comprising: (a) obtaining a sample from the patient; (b) administering the treatment compound to the sample; (c) determining the level of a biomarker in the sample; and (d) diagnosing the patient as being likely to be responsive to the treatment compound if the level of the biomarker in the sample is different from a reference level of the biomarker; wherein the treatment compound is Compound 1, or a pharmaceutically acceptable salt or a solvate thereof. In one embodiment, the biomarker is PNUTS.

In some embodiments of the methods provided herein, administering a treatment compound to the sample from the cancer patient is performed in vitro. In other embodiments, administering a treatment compound to the sample from the patient having cancer is performed in vivo. In certain embodiments, the sample is cells. In one embodiment, the cells are contacted with the treatment compound for a period of time, e.g., 5, 10, 15, 20, 25, 30, 35, 40, 45, 50, or 55 minutes, or 1, 2, 3, 4, 5, 6, 7, 8, 9, 10, 11, 12, 13, 14, 15, 16, 17, 18, 19, 20, 21, 22, 23, or 24 hours, or 2, 3, or more days. In other embodiments, the cells are obtained from another cancer patient.

In some embodiments, the level of the biomarker in the sample of the patient is higher than the reference level of the biomarker. In other embodiments, the level of the biomarker in the sample of the patient is lower than the reference level of the biomarker.

In yet another aspect, provided herein are methods of predicting the responsiveness of a cancer patient to a treatment compound, comprising: (a) administering the treatment compound to the patient; (b) obtaining a sample from the patient; (c) determining the level of PNUTS, ANK1, IBA57, SOWAHD, MF12-AS1, or CECR1 in the sample; (d) diagnosing the patient as being likely to be responsive to a treatment of the cancer with the treatment compound if the level of PNUTS, ANK1, IBA57, SOWAHD, MF12-AS1, or CECR1 in the sample is different than the level of PNUTS, ANK1, IBA57, SOWAHD, MF12-AS1, or CECR1 obtained from a reference sample; wherein the treatment compound is Compound 1, or a pharmaceutically acceptable salt or a solvate thereof. In one embodiment, the biomarker is PNUTS.

In yet another aspect, provided herein is a method of predicting the responsiveness of a cancer patient to a treatment compound, comprising: (a) obtaining a sample from the patient; (b) administering the treatment compound to the sample; (c) determining the level of PNUTS, ANK1, IBA57, SOWAHD, MF12-AS1, or CECR1 in the sample; and (d) diagnosing the patient as being likely to be responsive to a treatment of the cancer with the treatment compound if the level of PNUTS, ANK1, IBA57, SOWAHD, MF12-AS1, or CECR1 in the sample is different than the level of PNUTS, ANK1, IBA57, SOWAHD, MF12-AS1, or CECR1 obtained from a reference sample; wherein the treatment compound is Compound 1, or a pharmaceutically acceptable salt or a solvate thereof. In one embodiment, the level of PNUTS is determined and compared.

In yet another aspect, provided herein are methods of monitoring the efficacy of a treatment of cancer in a subject with a treatment compound, comprising: (a) administering the treatment compound to the patient; (b) obtaining a sample from the patient; (c) determining the level of PNUTS, ANK1, IBA57, SOWAHD, MF12-AS1, or CECR1 in the sample; (d) diagnosing the patient as being likely to be responsive to a treatment of the cancer with the treatment compound if the level of PNUTS, ANK1, IBA57, SOWAHD, MF12-AS1, or CECR1 in the sample is different than the level of PNUTS, ANK1, IBA57, SOWAHD, MF12-AS1, or CECR1 obtained from a reference sample; wherein the treatment compound is Compound 1, or a pharmaceutically acceptable salt or a solvate thereof. In some embodiments, a change in the level of the biomarker as compared to the reference is indicative of the efficacy of the treatment compound in treating the cancer in the patient. In one embodiment, an increased level of the biomarker is indicative of the efficacy of the treatment compound in treating the cancer in the patient. In another embodiment, a decreased level of the biomarker is indicative of the efficacy of the treatment compound in treating the cancer in the patient. In one embodiment, the level of PNUTS is determined and compared.

In certain embodiments, when the cancer patient is diagnosed as being likely to be responsive to the treatment compound, the methods provided herein further comprise administering a therapeutically effective amount of the treatment compound to the patient.

In another aspect, provided herein are methods of treating cancer, comprising: (a) obtaining a sample from a patient having the cancer; (b) determining the level of a biomarker in the sample; (c) diagnosing the patient as being likely to be responsive to a treatment compound if the level of the biomarker in the sample is different from a reference level of the biomarker; and (d) administering a therapeutically effective amount of the treatment compound to the patient; wherein the treatment compound is Compound 1, or a pharmaceutically acceptable salt or a solvate thereof. In one embodiment, the biomarker is PNUTS.

In some embodiments, the level of the biomarker in the sample of the patient is higher than the reference level of the biomarker. In other embodiments, the level of the biomarker in the sample of the patient is lower than the reference level of the biomarker.

In some embodiments of the various methods provided herein, a treatment compound is administered to a patient likely to be responsive to the treatment compound. Also provided herein are methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

As shown in the Examples, the levels of certain biomarkers change in response to Compound 1 treatment, such as PNUTS (PPP1R10), ANK1, IBA57, SOWAHD, MF12-AS1, and CECR1. Thus, in some embodiments, the biomarker is selected from the group consisting of PNUTS (PPP1R10), ANK1, IBA57, SOWAHD, MF12-AS1, and CECR1. In one embodiment, the biomarker is PNUTS (PPP1R10). In another embodiment, the biomarker is ANK1. In yet another embodiment, the biomarker is IBA57. In yet another embodiment, the biomarker is SOWAHD. In yet another embodiment, the biomarker is MF12-AS1. In yet another embodiment, the biomarker is CECR1.

In some embodiments of the various methods provided herein, the biomarker is PNUTS, ANK1, or IBA57, and wherein the level of PNUTS, ANK1, or IBA57 decreases as compared to a reference in response to Compound 1 treatment. In some embodiments of the various methods provided herein, the biomarker is SOWAHD, MF12-AS1, or CECR1 and the level of SOWAHD, MF12-AS1, or CECR1 increases as compared to a reference in response to Compound 1 treatment.

In certain embodiments, the various methods provided herein use samples (e.g., biological samples) from subjects or individuals (e.g., patients). The patient can be a patient, such as, a patient with a cancer (e.g., colorectal cancer). The patient can be male or female, and can be an adult, a child, or an infant. Samples can be analyzed at a time during an active phase of a cancer (e.g., colorectal cancer), or when the cancer (e.g., colorectal cancer) is inactive. In certain embodiments, more than one sample from a patient can be obtained.

In some embodiments, the sample used in the present methods comprises a biopsy (e.g., a tumor biopsy). The biopsy can be from any organ or tissue, for example, skin, liver, lung heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the patient prior to the patient receiving a treatment for the disease or disorder. In another embodiment, the sample is obtained from the patient during the patient receiving a treatment for the disease or disorder. In another embodiment, the sample is obtained from the patient after the patient has received a treatment for the disease or disorder. In various embodiments, the treatment comprises administering Compound 1 to the patient.

In certain embodiments, the sample used in the methods provided herein comprises a plurality of cells, such as cancer (e.g., colorectal cancer) cells. Such cells can include any type of cells, e.g., stem cells, blood cells (e.g., peripheral blood mononuclear cells (PBMC)), lymphocytes, B cells, T cells, monocytes, granulocytes, immune cells, or cancer cells.

### Pharmaceutical Compositions

In certain embodiments, the heterocyclic KDM4 inhibitor described herein is administered as a pure chemical. In other embodiments, the heterocyclic KDM4 inhibitor described herein is combined with a pharmaceutically suitable or acceptable carrier (also referred to herein as a pharmaceutically suitable or acceptable excipient, a physiologically suitable or acceptable excipient, or a physiologically suitable or acceptable carrier) selected on the basis of a chosen route of administration and standard pharmaceutical practice.

Provided herein is a pharmaceutical composition comprising the heterocyclic KDM4 inhibitor as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a method of preparing a pharmaceutical composition comprising mixing the heterocyclic KDM4 inhibitor as described herein, or a stereoisomer, pharmaceutically acceptable salt, hydrate, or solvate thereof, and a pharmaceutically acceptable carrier.

Provided herein is the method wherein the pharmaceutical composition is administered orally. Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

Provided herein is the method wherein the pharmaceutical composition is administered by injection. In some embodiments, the heterocyclic KDM4 inhibitor as described herein, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising the heterocyclic KDM4 inhibitor as described herein, or a stereoisomer, pharmaceutically acceptable salt, hydrate, or solvate thereof, differs depending upon the subject or patient's (e.g., human) condition. In some embodiments, such factors include general health status, age, and other factors. Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

### EXAMPLES

These examples are provided for illustrative purposes only and not to limit the scope of the claims provided herein.

### Background

The KDM4 family of histone lysine demethylases consists of four main isoforms (KDM4A, B, C, D), all of which have been identified as key oncogenic drivers. They function as epigenetic regulators and control transitions between transcriptionally silent and active chromatin states via removal of methyl marks on histone H3K9 and histone H3K36.KDM4 isoforms play an important role in the epigenetic dysregulation in various cancers and is linked to more aggressive disease and poorer clinical outcomes. Functional redundancy and cross-activity have been observed across KDM4 family members, thus, selective inhibition of one isoform appears to not be effective. Compound 1 is a pan inhibitor of KDM4 that simultaneously targets multiple isoforms of KDM4.

### Example 1: KDM4 Inhibitory Activity of Compound 1

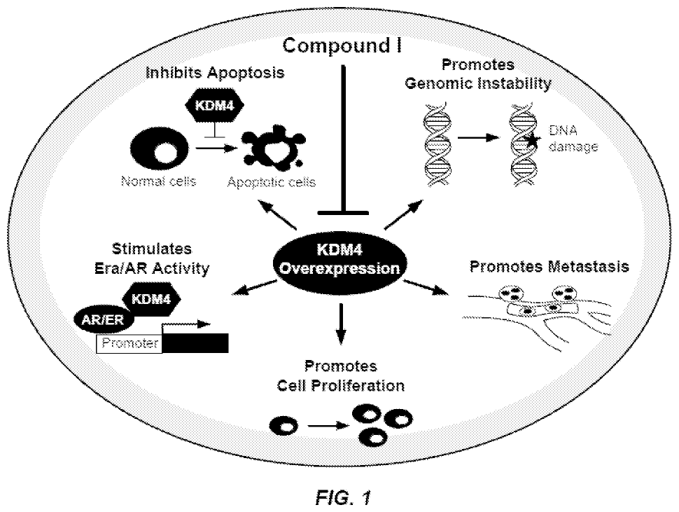

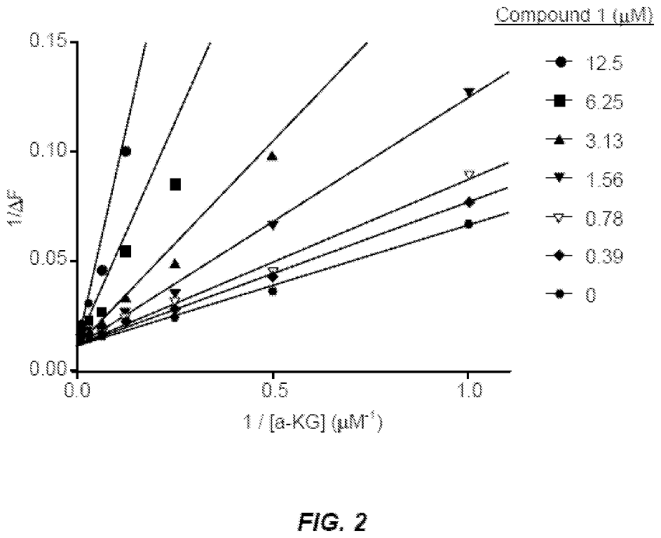

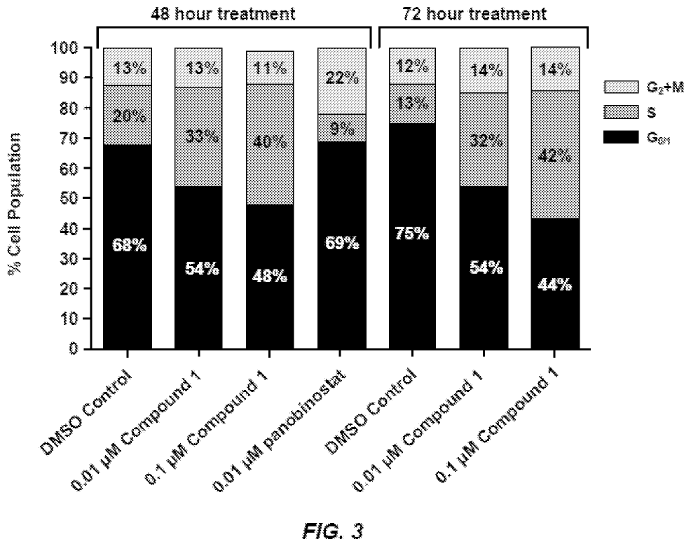

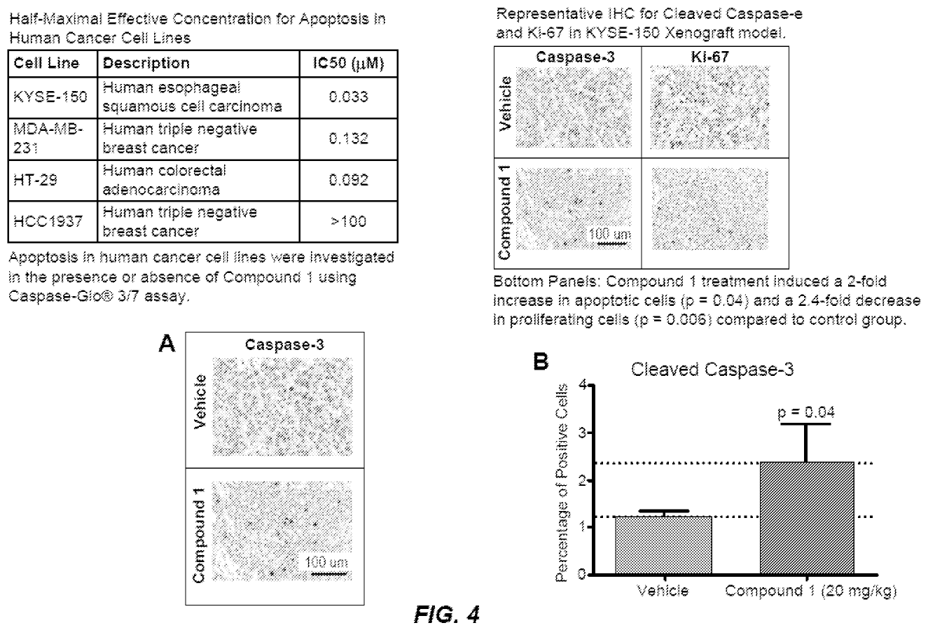

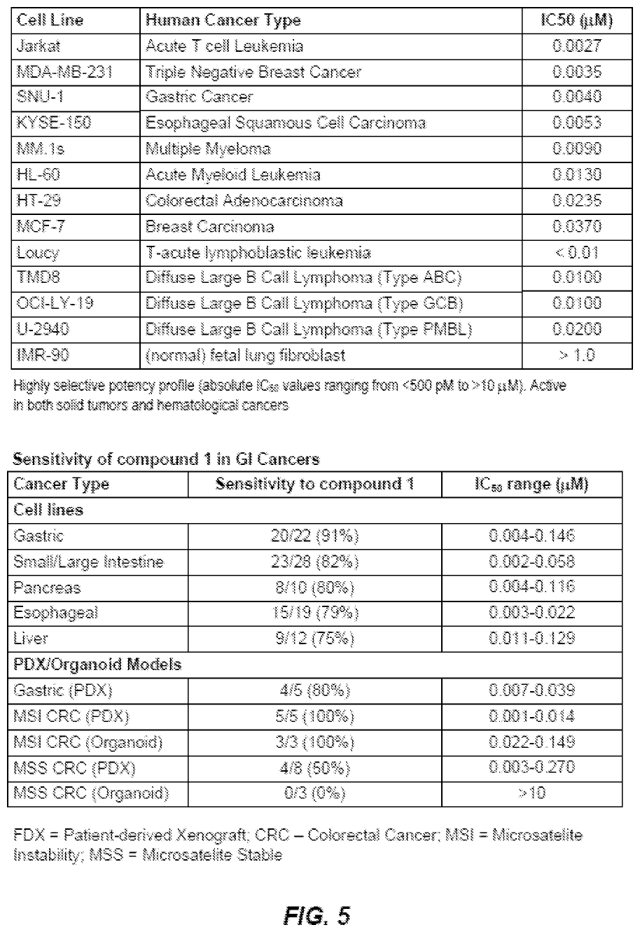

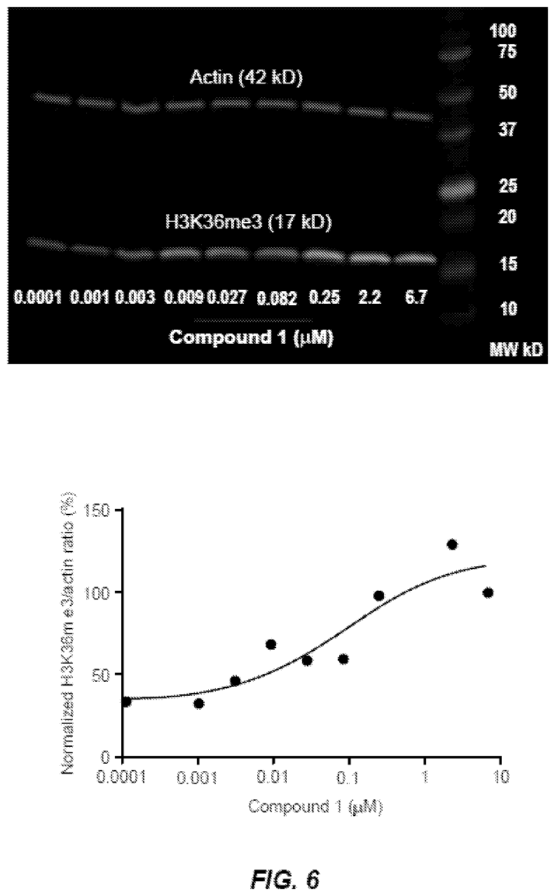

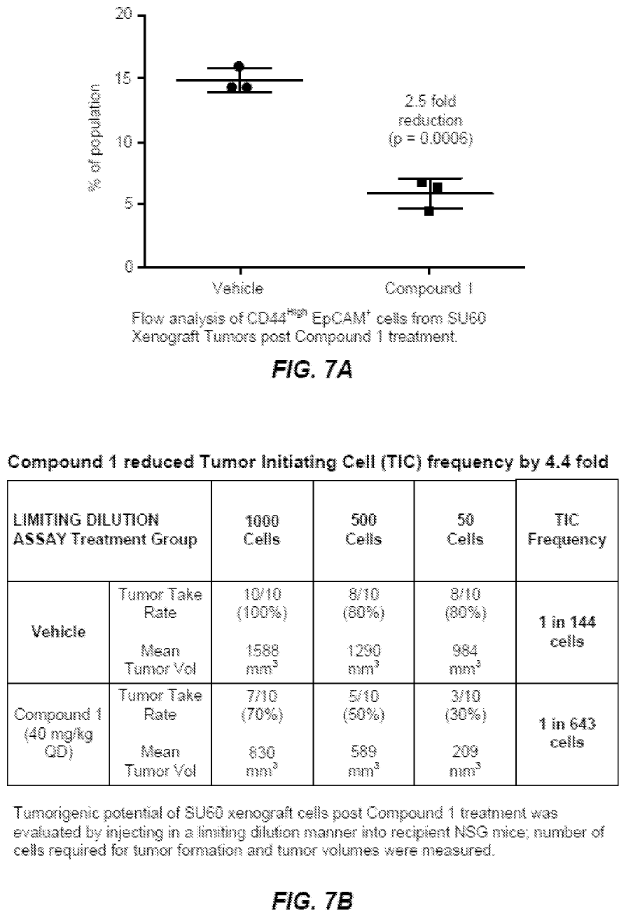

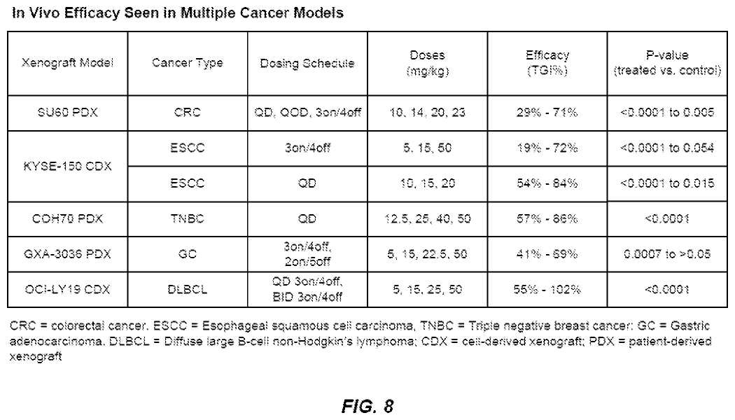

FIG. 1 illustrates the biochemical activities of Compound 1, a potent PAN KDM4 inhibitor. FIG. 2 illustrates the reversible and competitive inhibition of H3K9me3 demethylation. FIG. 3 illustrates that compound 1 induces cell cycle arrest in S-phase. FIG. 4 demonstrates that Compound 1 induces apoptosis in human cancer cell lines. See also: PCT patent publication WO2015/200709 and related patent applications and granted patents, such as U.S. Pat. No. 9,242,968, which are incorporated by reference in their entirety.

### Example 2a: Compound 1 Inhibits Cell Proliferation in Cell Culture and Organoid Models

Compound 1 was evaluated in in vitro cell-proliferation assays (BrdU assay, MTS assay, or Cell Titer Glo (CTG) assay) in multiple cancer cell lines and patient-derived organoid models to determine half-maximal inhibitory concentrations (IC50s).

Viability and BrdU thymidine incorporation assays were performed in multiwell plates seeded with cells in growth medium. After seeding, multiwell plates were cultured for 24 hours in a humidified incubator at 37° C. to promote adherence. The assay was initiated in individual test wells by adding either DMSO as a negative control, or an inhibitor panobinostat at ≥100×IC50 concentration as a positive control, or serially diluted Compound 1. The cultures were incubated for 168 hours after which the number of viable cells or the amount of thymidine incorporation in each test well was assessed by the methods as indicated above. Readouts were performed using an EnVision® Multilabel Reader (PerkinElmer, Waltham, MA) and the results were expressed as a percent of the negative control. Percent of Control values were plotted against the corresponding Compound 1 concentration and the relative IC50 value was determined from a non-linear regression curve as the concentration where inhibition was half-maximal. For cell lines where the number of IC50 determinations exceeded one, the IC50 was expressed as the mean IC50±SD.

Half-maximal inhibitory concentration (IC50) values for Compound 1 inhibition of cell proliferation were determined for nine cancer cell lines and one normal human fibroblastcell line. As shown in Table 1, except for IMR-90, a normal human fibroblastline, and ZR-75-1, a human breast carcinoma line, Compound 1 treatment for 168 hours demonstrated potent anti-proliferative activity across the panel, which included both solid and hematological cancer cell lines. Compound 1 showed potent anti-proliferative activity (<0.040 μM) for eight solid and hematological cancer cell lines: Jurkat, MDA-MB-231, KYSE-150, MM.1 s, HL-60, HT-29, MCF-7, and Loucy.

TABLE 1

| | | | | | Assay Format | | |
|---|---|---|---|---|---|---|---|
| Cell Line | Description | IC₅₀ (μM) | SD (μM) | N(n) | BrdU | MTS | CTG |
| Jurkat | human acute T cell leukemia | 0.0027 | 0.0022 | 4(2) | 1 | 2 | 1 |
| MDA-MB-231 | human triple negative breast cancer | 0.0035 | 0.0050 | 4(3) | 4 | — | — |
| KYSE-150 | human esophageal squamous cell carcinoma | 0.0053 | 0.0040 | 8(3) | 5 | 3 | — |
| MM.1s | human multiple myeloma | 0.0090 | — | 1(1) | — | 1 | — |
| HL-60 | human acute myeloid leukemia | 0.0130 | — | 1(1) | — | 1 | — |
| HT-29 | human colorectal adenocarcinoma | 0.0235 | 0.0134 | 2(2) | 1 | — | 1 |
| MCF-7 | human breast carcinoma | 0.0370 | — | 1(1) | — | 1 | — |
| Loucy | human T-acute lymphoblastic leukemia | <0.01 | — | 1(1) | — | 1 | — |
| IMR-90 | (normal) human fetal lung fibroblast | >1.0 | — | 2(1) | 1 | — | 1 |
| ZR-75-1 | human breast carcinoma | >10 | — | 1(1) | — | 1 | — |

MTS = CellTiter 96 ® AQueous One Solution Cell Proliferation Assay, CTG = CellTiter-Glo ® Luminescent Cell Viability Assay, and BrdU = 5-bromo-2-deoxyuridine thymidine incorporation assay where the number indicates how many of each assay was performed.

The IC₅₀ values for Compound 1 inhibition of colony formation and viability were determined for the patient-derived colon cancer organoid models: SU60, T002C, SU62, SU34, SU103, T03SC, and SU106, patient-derived pancreatic cancer organoid models PA016SF, PA0143F, T016P, and T028P, and a patient-derived breast cancer organoid model FS5S3.

Colony formation and viability assays were performed in multiwell plates seeded with organoids using an in vitro culture system composed of a Matrigel layer, media containing a defined set of growth factors, and co-culture with Wnt3 A-secreting mouse embryonic fibroblasts. Assays were initiated by adding either DMSO as a negative control, or an inhibitor panobinostatat 100×IC50 concentration as a positive control, or serially diluted Compound 1. The cultures were incubated for 168 or 144 hours after which the number of viable organoids in each test well was assessed respectively using one of two detection methods: CTG or staining with calcein-AM followed by imaging and colony counting. Colony count or viability of cells treated with compound 1 was expressed as a percent of the negative control. Aggregated Percent of Control values were plotted against the corresponding Compound 1 concentration and the absolute IC50 value was determined from a non-linear regression curve as the concentration where inhibition was 50% of the control.

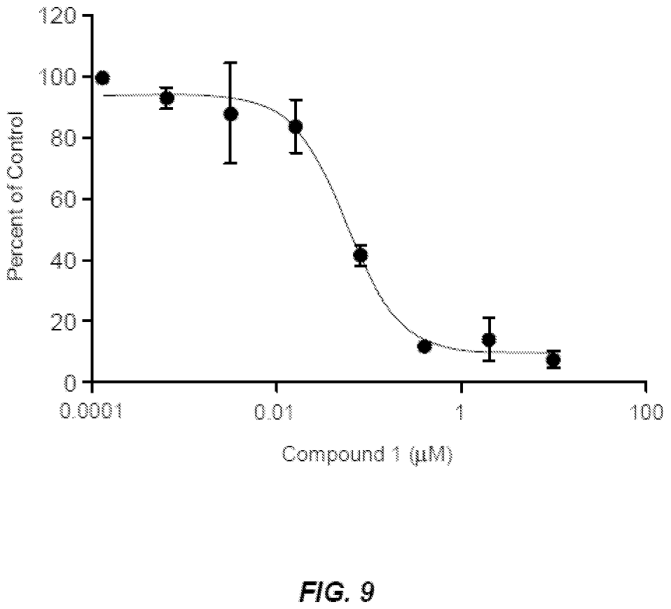

As shown in Table 2, Compound 1 treatment demonstrated sub-micromolar anti-proliferative activity for the colorectal carcinoma models SU60, T002C, and SU62 yielding IC50 values<0.15 M. Compound 1 treatment also demonstrated potent anti-proliferative activity for the PA0165F pancreatic carcinoma model yielding IC50 values<0.03 M. The SU34, SU103, T035C, and SU106 colorectal carcinoma models and the pancreatic carcinoma models PA0143F, T016P, and T028P were unresponsive to Compound 1 in these colony formation and viability assays with IC50 values>10 M. An IC50 value of 13.6 M was determined for the FS53 human breast carcinoma model following compound 1 treatment. A representative titration curve for data aggregated across two SU60 assays was constructed and presented as FIG. 9.

TABLE 2

| | Organoid | | | Assay Format | |
|---|---|---|---|---|---|
| Description | Model | IC₅₀ (μM) | N | Time (hrs) | Readout |
| human | SU60 | 0.023 | 2 | 168 | CTG |
| colorectal | | 0.064 | 2 | 144 | Colony Counts |
| carcinoma | T002C | 0.022 | 4 | 144 | Colony Counts |
| | SU62 | 0.149 | 2 | 144 | Colony Counts |
| | SU34 | >10 | 4 | 144 | Colony Counts |
| | SU103 | >10 | 3 | 144 | Colony Counts |
| | T035C | >10 | 3 | 144 | Colony Counts |
| | SU106 | >10 | 1 | 144 | Colony Counts |
| human | PA0165F | 0.029 | 1 | 168 | CTG |
| pancreatic | | 0.016 | 5 | 144 | Colony Counts |
| carcinoma | PA0143F | >10 | 4 | 144 | Colony Counts |
| | T016P | >10 | 4 | 144 | Colony Counts |
| | T028P | >10 | 6 | 144 | Colony Counts |
| human breast | FS53 | 13.6 | 2 | 144 | Colony Counts |
| carcinoma | | | | | |

As shown in FIG. 9, percent of control=colony counts for Compound 1 treated organoids normalized to negative control colony counts, expressed as a percent; the curve combined data from two SU60 experiments; error bars are ±SD for two independent experiments. The colony formation inhibition assays were performed in multiwell plates seeded with SU60 organoids. The cultures were incubated for 168 hours after which the number of colonies was assessed by staining with calcein AM followed by imaging and colony counting. Colony counts were expressed as a percent of the negative control and aggregated Percent of Control values were plotted against the corresponding Compound 1 concentration. A curve was fitted using non-linear regression.

### Example 2b: Evaluation of Compound 1 in Cancer Cell Line Panel

In a larger screening study, compound 1 was evaluated across a broad cancer cell line panel (OncoPanel; HD Biosciences) composed of 301 cancer cell lines from different tumor types.

2D cell viability inhibition assays were performed in multiwell plates seeded with cells from the panel. After seeding, the plates were cultured overnight in a humidified incubator at 37° C. to promote adherence. Assays were initiated in individual wells by adding either DMSO as a vehicle control, growth media as a blank, or serially diluted compound 1 (10 μM to 0.0005 μM with 1:3 serial dilution). Cultures were incubated for 168 hours after which the number of viable cells in each test well was assessed using the CellTiter-Glo® Luminescent Cell Viability Assay. Luminescence readouts were performed using an EnVision® Multilabel Reader and the compound 1 readouts were normalized to the DMSO control readouts, expressed as a percent of the control. Outliers were flagged out by visual inspection. Percent of control was plotted against the corresponding compound 1 concentration and the absolute IC50 value was determined using four-parameter logistic non-linear regression as the concentration where inhibition was 50% of the control. For maximum inhibition<50%, the absolute IC50 was reported as >10. In addition to EC50 and IC50, AUC was determined and was normalized to the area corresponding to theoretical no-inhibition (the rectangular area defined by the compound dose range and 0 to 100 in y). Emax was recorded as the minimum in y with the compound dose range. For those cell lines where Emax>40, EC50 was manually set to 10 M. A hierarchical clustering algorithm based on Euclidean distance between the standardized input values and “Ward.D” method as implemented in R hclust function was used to visualize the compound 1 cellular potency data. The sensitive/moderate/resistant calls for each cell line was determined by visually inspecting the resulting clustering dendrogram. All the analyses were done in R Core Team.

Results demonstrated that compound 1 potently inhibited proliferation of cancer cell lines and displayed a highly selective potency profile with absolute IC₅₀ values ranging from <500 pM to >10 μM. Among the 301 cell lines, 209 were inhibited by compound 1 with IC₅₀ values<1 μM while 89 lines were insensitive with IC₅₀ values>10 μM. Table 3 provides the results for all 301 cell lines.

TABLE 3

| Cell line | IC₅₀(μM) | EC₅₀(μM) | AUC | Emax | Tumor Type | Tumor Subtype |
|---|---|---|---|---|---|---|
| NCIH1618 | 5.00E−04 | 0.000508 | 0.022662 | 0.488613 | lung | small cell carcinoma |
| NCIH1048 | 0.000706 | 0.000508 | 0.147882 | 7.150228 | lung | small cell carcinoma |
| NCIH1155 | 0.000765 | 0.000884 | 0.075713 | 0.26377 | lung | large cell carcinoma |
| NCIH1666 | 0.001165 | 0.000508 | 0.298302 | 21.65869 | lung | bronchioloalveolar |
| | | | | | | adenocarcinoma |
| REH | 0.00121 | 0.001324 | 0.095695 | 0.233625 | haematopoietic | acute lymphoblastic B cell |
| | | | | | and lymphoid | leukaemia |
| | | | | | tissue | |
| MOLT4 | 0.00125 | 0.000508 | 0.137568 | 0.604781 | haematopoietic | acute lymphoblastic T cell |
| | | | | | and lymphoid | leukaemia |
| | | | | | tissue | |
| NCIH209 | 0.001307 | 0.00148 | 0.186896 | 8.610089 | lung | small cell carcinoma |
| JURKAT | 0.001488 | 0.001655 | 0.117145 | 0.211954 | haematopoietic | acute lymphoblastic T cell |
| | | | | | and lymphoid | leukaemia |
| | | | | | tissue | |
| NCIH1694 | 0.001753 | 0.000508 | 0.321443 | 21.59348 | lung | small cell carcinoma |
| HCT15 | 0.001791 | 0.001473 | 0.183818 | 4.540784 | large intestine | adenocarcinoma |
| JEKO1 | 0.001804 | 0.002309 | 0.117241 | 0.087378 | haematopoietic | mantle cell lymphoma |
| | | | | | and lymphoid | |
| | | | | | tissue | |
| TALL1 | 0.001805 | 0.000895 | 0.199872 | 4.509292 | haematopoietic | acute lymphoblastic T cell |
| | | | | | and lymphoid | leukaemia |
| | | | | | tissue | |
| A2780 | 0.002137 | 0.002099 | 0.181084 | 3.567055 | ovary | adenocarcinoma |
| LOUCY | 0.002474 | 0.002471 | 0.188038 | 2.522241 | haematopoietic | acute lymphoblastic T cell |
| | | | | | and lymphoid | leukaemia |
| | | | | | tissue | |
| DLD1 | 0.002533 | 0.001467 | 0.226049 | 5.540107 | large intestine | adenocarcinoma |
| U937 | 0.002554 | 0.002732 | 0.180761 | 1.946235 | haematopoietic | diffuse large B cell |
| | | | | | and lymphoid | lymphoma |
| | | | | | tissue | |
| NCIH2081 | 0.002568 | 0.001459 | 0.304851 | 17.9809 | lung | small cell carcinoma |
| TF1 | 0.002586 | 0.00158 | 0.288323 | 11.54969 | haematopoietic | acute myeloid leukaemia |
| | | | | | and lymphoid | |
| | | | | | tissue | |
| RL | 0.002993 | 0.001877 | 0.236617 | 1.95413 | haematopoietic | B cell lymphoma |
| | | | | | and lymphoid | unspecified |
| | | | | | tissue | |
| NCIH2023 | 0.003031 | 0.002842 | 0.288394 | 13.34318 | lung | adenocarcinoma |
| NAMALWA | 0.003206 | 0.003265 | 0.24309 | 6.45672 | haematopoietic | Burkitt lymphoma |
| | | | | | and lymphoid | |
| | | | | | tissue | |
| TE14 | 0.003393 | 0.002324 | 0.306836 | 12.67238 | oesophagus | squamous cell carcinoma |
| KYSE510 | 0.003395 | 0.003418 | 0.199839 | 0.579743 | oesophagus | squamous cell carcinoma |
| DMS79 | 0.003467 | 0.00128 | 0.384485 | 27.09377 | lung | small cell carcinoma |
| NCIH2286 | 0.003508 | 0.00253 | 0.291713 | 11.19933 | lung | small cell carcinoma |
| NCIH82 | 0.003543 | 0.003315 | 0.250228 | 4.909701 | lung | small cell carcinoma |
| NCIH2087 | 0.003555 | 0.002583 | 0.382152 | 21.97574 | lung | adenocarcinoma |
| PF382 | 0.003685 | 0.001894 | 0.276144 | 8.404679 | haematopoietic | acute lymphoblastic T cell |
| | | | | | and lymphoid | leukaemia |
| | | | | | tissue | |
| YD38 | 0.003771 | 0.003547 | 0.298266 | 13.63878 | upper | squamous cell carcinoma |
| | | | | | aerodigestive tract | |
| HPAC | 0.003928 | 0.002376 | 0.396472 | 20.84918 | pancreas | ductal carcinoma |
| PECAPJ34CLONEC12 | 0.003966 | 0.003807 | 0.311032 | 11.80269 | upper | squamous cell carcinoma |
| | | | | | aerodigestive tract | |
| MV411 | 0.004017 | 0.003916 | 0.216311 | 0.056467 | haematopoietic | acute myeloid leukaemia |
| | | | | | and lymphoid | |
| | | | | | tissue | |
| AZ521 | 0.004189 | 0.003845 | 0.287487 | 8.891497 | small intestine | NS |
| SNU16 | 0.004217 | 0.003693 | 0.282065 | 7.431244 | stomach | undifferentiated |
| | | | | | | adenocarcinoma |
| TE11 | 0.00428 | 0.003854 | 0.304041 | 10.15582 | oesophagus | squamous cell carcinoma |
| NCIN87 | 0.004292 | 0.003935 | 0.313195 | 10.80159 | stomach | NS |
| P31FUJ | 0.004324 | 0.002434 | 0.285987 | 7.305396 | haematopoietic | acute myeloid leukaemia |
| | | | | | and lymphoid | |
| | | | | | tissue | |
| CORL279 | 0.004345 | 0.004231 | 0.315531 | 11.3227 | lung | small cell carcinoma |
| HUTU80 | 0.004361 | 0.004208 | 0.301592 | 8.150503 | small intestine | adenocarcinoma |
| HCC95 | 0.0044 | 0.003342 | 0.407204 | 17.03582 | lung | squamous cell carcinoma |
| RT11284 | 0.004435 | 0.00414 | 0.371749 | 18.19969 | urinary tract | NS |
| G401 | 0.004449 | 0.004392 | 0.250793 | 3.80796 | soft tissue | NS |
| K562 | 0.004556 | 0.004083 | 0.274996 | 4.382836 | haematopoietic | blast phase chronic myeloid |
| | | | | | and lymphoid | leukaemia |
| | | | | | tissue | |
| MC116 | 0.004605 | 0.005215 | 0.252694 | 5.430331 | haematopoietic | B cell lymphoma |
| | | | | | and lymphoid | unspecified |
| | | | | | tissue | |
| COLO320HSR | 0.004609 | 0.00446 | 0.240303 | 0.900662 | large intestine | adenocarcinoma |
| LNCAP | 0.004648 | 0.003545 | 0.397386 | 21.5094 | prostate | adenocarcinoma |
| NOMO1 | 0.005012 | 0.003922 | 0.329287 | 8.171852 | haematopoietic | acute myeloid leukaemia |
| | | | | | and lymphoid | |
| | | | | | tissue | |
| KYSE150 | 0.005097 | 0.004407 | 0.305205 | 9.556776 | oesophagus | squamous cell carcinoma |
| KYSE270 | 0.005351 | 0.005115 | 0.306237 | 7.792632 | oesophagus | squamous cell carcinoma |
| C32 | 0.00536 | 0.005182 | 0.253167 | 1.007096 | skin | NS |
| SKMEL28 | 0.005562 | 0.005627 | 0.279597 | 4.919564 | skin | NS |
| WSUDLCL2 | 0.005671 | 0.004215 | 0.282886 | 0.634625 | haematopoietic | diffuse large B cell |
| | | | | | and lymphoid | lymphoma |
| | | | | | tissue | |
| MKN1 | 0.005748 | 0.004335 | 0.39452 | 17.75546 | stomach | mixed adenosquamous |
| | | | | | | carcinoma |
| NCIH520 | 0.005895 | 0.005059 | 0.323681 | 10.58477 | lung | squamous cell carcinoma |
| NCIH1836 | 0.006075 | 0.004881 | 0.333178 | 10.40721 | lung | small cell carcinoma |
| CHL1 | 0.006077 | 0.004679 | 0.349333 | 13.21706 | skin | NS |
| THP 1 | 0.00615 | 0.004924 | 0.337139 | 12.47527 | haematopoietic | acute myeloid leukaemia |
| | | | | | and lymphoid | |
| | | | | | tissue | |
| AGS | 0.006186 | 0.005182 | 0.3536 | 14.1129 | stomach | adenocarcinoma |
| TOV112D | 0.006227 | 0.004264 | 0.3964 | 23.18819 | ovary | endometrioid carcinoma |
| CAOV3 | 0.006468 | 0.005254 | 0.390842 | 19.3273 | ovary | adenocarcinoma |
| KARPAS422 | 0.006525 | 0.005425 | 0.328997 | 8.084201 | haematopoietic | diffuse large B cell |
| | | | | | and lymphoid | lymphoma |
| | | | | | tissue | |
| NCIH358 | 0.006571 | 0.003053 | 0.433186 | 22.65882 | lung | bronchioloalveolar |
| | | | | | | adenocarcinoma |
| TE6 | 0.006677 | 0.004833 | 0.362521 | 14.02411 | oesophagus | squamous cell carcinoma |
| T.T | 0.006752 | 0.004366 | 0.432105 | 24.08447 | oesophagus | squamous cell carcinoma |
| NCIH524 | 0.006853 | 0.004977 | 0.361681 | 12.7766 | lung | small cell carcinoma |
| TE9 | 0.00689 | 0.003749 | 0.424717 | 25.30919 | oesophagus | squamous cell carcinoma |
| HSC4 | 0.006951 | 0.004332 | 0.43466 | 23.27648 | upper | squamous cell carcinoma |
| | | | | | aerodigestive tract | |
| MIAPACA2 | 0.007021 | 0.005283 | 0.332162 | 9.710695 | pancreas | ductal carcinoma |
| NCIH69 | 0.007089 | 0.00714 | 0.280211 | 1.270819 | lung | small cell carcinoma |
| KM12 | 0.007345 | 0.006303 | 0.348209 | 12.23928 | large intestine | adenocarcinoma |
| DV90 | 0.007364 | 0.005355 | 0.369777 | 11.78178 | lung | adenocarcinoma |
| FTC133 | 0.007491 | 0.004279 | 0.414551 | 22.3385 | thyroid | follicular carcinoma |
| NCIH1299 | 0.007498 | 0.005384 | 0.350839 | 12.64795 | lung | non-small cell carcinoma |
| NUGC3 | 0.00772 | 0.005627 | 0.38244 | 15.99752 | stomach | NS |
| NCIH1703 | 0.008047 | 0.004878 | 0.420393 | 21.7324 | lung | adenocarcinoma |
| KYSE410 | 0.008253 | 0.005469 | 0.415601 | 21.4639 | oesophagus | squamous cell carcinoma |
| NCIH2291 | 0.008405 | 0.007553 | 0.38223 | 13.48474 | lung | adenocarcinoma |
| TE5 | 0.008472 | 0.006638 | 0.387656 | 15.07094 | oesophagus | squamous cell carcinoma |
| U2OS | 0.009007 | 0.007712 | 0.406496 | 9.064236 | bone | NS |
| SW1463 | 0.009244 | 0.009177 | 0.340776 | 7.126189 | large intestine | adenocarcinoma |
| HL60 | 0.009317 | 0.008115 | 0.370342 | 9.446155 | haematopoietic | acute myeloid leukaemia |
| | | | | | and lymphoid | |
| | | | | | tissue | |
| NCIH716 | 0.009514 | 0.005771 | 0.379883 | 12.75198 | large intestine | adenocarcinoma |
| NCIH661 | 0.009732 | 0.009063 | 0.360829 | 9.074949 | lung | large cell carcinoma |
| SW480 | 0.009788 | 0.007726 | 0.409035 | 20.35763 | large intestine | adenocarcinoma |
| BFTC905 | 0.009914 | 0.008929 | 0.39887 | 13.73926 | urinary tract | transitional cell carcinoma |
| BT549 | 0.009983 | 0.005837 | 0.431039 | 20.54694 | breast | ductal carcinoma |
| NCIH508 | 0.010235 | 0.009159 | 0.337789 | 3.207058 | large intestine | adenocarcinoma |
| KU1919 | 0.010268 | 0.008773 | 0.395587 | 14.57044 | urinary tract | transitional cell carcinoma |
| COLO741 | 0.01036 | 0.006858 | 0.418613 | 18.78304 | skin | NS |
| MDAMB453 | 0.010402 | 0.008034 | 0.452361 | 21.95264 | breast | NS |
| LU65 | 0.010583 | 0.00719 | 0.409074 | 11.99716 | lung | non-small cell carcinoma |
| A375 | 0.010596 | 0.007623 | 0.440543 | 19.51092 | skin | NS |
| HCC70 | 0.01099 | 0.008561 | 0.413475 | 13.84525 | breast | ductal carcinoma |
| A101D | 0.011003 | 0.006914 | 0.468887 | 20.45562 | skin | NS |
| HLE | 0.011279 | 0.010386 | 0.383928 | 7.875661 | liver | hepatocellular carcinoma |
| RKO | 0.011534 | 0.008496 | 0.382689 | 11.05361 | large intestine | adenocarcinoma |
| NCIH460 | 0.011582 | 0.01108 | 0.371384 | 7.704677 | lung | large cell carcinoma |
| SNB19 | 0.011607 | 0.007011 | 0.467719 | 20.73493 | central nervous | astrocytoma Grade IV |
| | | | | | system | |
| HUH1 | 0.011768 | 0.010591 | 0.414451 | 11.68072 | liver | hepatocellular carcinoma |
| KMS11 | 0.012224 | 0.01094 | 0.391826 | 9.982711 | haematopoietic | plasma cell myeloma |
| | | | | | and lymphoid | |
| | | | | | tissue | |
| 647V | 0.01224 | 0.01115 | 0.391223 | 10.17987 | urinary tract | transitional cell carcinoma |
| TE4 | 0.012665 | 0.009106 | 0.394771 | 11.49063 | oesophagus | squamous cell carcinoma |
| NCIH1437 | 0.013132 | 0.009476 | 0.408233 | 14.97097 | lung | adenocarcinoma |
| HUH7 | 0.013955 | 0.012904 | 0.392615 | 7.054246 | liver | hepatocellular carcinoma |
| 143B | 0.014366 | 0.013944 | 0.358228 | 2.195792 | bone | NS |
| LS411N | 0.016662 | 0.016827 | 0.385144 | 6.414977 | large intestine | adenocarcinoma |
| SKMEL5 | 0.016833 | 0.014822 | 0.407428 | 8.227588 | skin | NS |
| KYSE30 | 0.002426 | 10 | 0.44339 | 38.58605 | oesophagus | squamous cell carcinoma |
| CAKI1 | 0.004429 | 10 | 0.531935 | 34.02825 | kidney | clear cell renal cell |
| | | | | | | carcinoma |
| HCC1599 | 0.005645 | 0.003823 | 0.463913 | 32.70157 | breast | ductal carcinoma |
| 5637 | 0.006107 | 0.002239 | 0.485659 | 33.00819 | urinary tract | NS |
| HCC1187 | 0.006507 | 0.003097 | 0.464968 | 33.99519 | breast | ductal carcinoma |
| COLO680N | 0.006972 | 0.004787 | 0.531063 | 33.76189 | oesophagus | squamous cell carcinoma |
| SKBR3 | 0.007137 | 0.004323 | 0.439603 | 26.58705 | breast | NS |
| NCIH1092 | 0.00716 | 0.003931 | 0.479044 | 26.40383 | lung | small cell carcinoma |
| OCUM1 | 0.00779 | 10 | 0.532422 | 32.42435 | stomach | diffuse adenocarcinoma |
| HSC2 | 0.007994 | 0.005088 | 0.451097 | 26.00789 | upper | squamous cell carcinoma |
| | | | | | aerodigestive tract | |
| NCIH1436 | 0.008754 | 10 | 0.495332 | 34.40202 | lung | small cell carcinoma |
| SKMEL1 | 0.009242 | 0.000536 | 0.442658 | 22.0664 | skin | NS |
| RD | 0.009294 | 0.004792 | 0.470348 | 27.41572 | soft tissue | embryonal |
| NCIH647 | 0.009841 | 0.006045 | 0.494182 | 28.28713 | lung | mixed adenosquamous |
| | | | | | | carcinoma |
| SCC4 | 0.010532 | 0.005568 | 0.461894 | 26.07786 | upper | squamous cell carcinoma |
| | | | | | aerodigestive tract | |
| NUGC4 | 0.010595 | 0.006625 | 0.464202 | 24.52784 | stomach | signet ring adenocarcinoma |
| HMCB | 0.011093 | 0.000815 | 0.504089 | 32.24977 | skin | NS |
| COLO205 | 0.011489 | 0.005969 | 0.483296 | 27.13083 | large intestine | adenocarcinoma |
| HEL | 0.011746 | 0.003598 | 0.526792 | 28.88964 | haematopoietic | acute myeloid leukaemia |
| | | | | | and lymphoid | |
| | | | | | tissue | |
| RPMI8226 | 0.01249 | 0.010401 | 0.436254 | 12.45893 | haematopoietic | plasma cell myeloma |
| | | | | | and lymphoid | |
| | | | | | tissue | |
| SW780 | 0.013009 | 0.011796 | 0.449063 | 13.9761 | urinary tract | transitional cell carcinoma |
| HCC1806 | 0.013019 | 0.005129 | 0.532503 | 36.86894 | breast | ductal carcinoma |
| NCIH2347 | 0.01306 | 10 | 0.577758 | 40.28995 | lung | adenocarcinoma |
| SHSY5Y | 0.013453 | 0.00432 | 0.492725 | 25.03904 | autonomic ganglia | NS |
| MKN45 | 0.013529 | 0.011004 | 0.442717 | 19.77129 | stomach | diffuse adenocarcinoma |
| HS578T | 0.013853 | 0.006902 | 0.537295 | 32.74703 | breast | ductal carcinoma |
| HT1080 | 0.014147 | 0.01275 | 0.543789 | 29.73724 | soft tissue | NS |
| HT55 | 0.014167 | 0.012668 | 0.470447 | 22.172 | large intestine | adenocarcinoma |
| MESSA | 0.014307 | 0.009003 | 0.493277 | 24.21537 | soft tissue | NS |
| PC3 | 0.014471 | 0.011814 | 0.502021 | 26.1466 | prostate | adenocarcinoma |
| U87MG | 0.014695 | 10 | 0.514049 | 38.45109 | central nervous | astrocytoma |
| | | | | | system | |
| CALU6 | 0.014859 | 0.011452 | 0.459539 | 18.31843 | lung | undifferentiated carcinoma |
| DAUDI | 0.014888 | 0.013395 | 0.538295 | 25.4109 | haematopoietic | Burkitt lymphoma |
| | | | | | and lymphoid | |
| | | | | | tissue | |
| KNS62 | 0.014901 | 0.012375 | 0.47611 | 18.83614 | lung | squamous cell carcinoma |
| SCABER | 0.015202 | 0.008931 | 0.568701 | 32.32311 | urinary tract | transitional cell carcinoma |
| CFPAC1 | 0.015268 | 0.011256 | 0.52266 | 29.21979 | pancreas | ductal carcinoma |
| NCIH1793 | 0.015288 | 0.008364 | 0.491588 | 27.47757 | lung | non-small cell carcinoma |
| TE10 | 0.016273 | 0.009297 | 0.529321 | 29.64652 | oesophagus | squamous cell carcinoma |
| NCIH1944 | 0.016307 | 10 | 0.580862 | 37.59181 | lung | non-small cell carcinoma |
| DETROIT562 | 0.016685 | 0.010403 | 0.587348 | 36.50766 | upper | NS |
| | | | | | aerodigestive tract | |
| NCIH522 | 0.01739 | 0.011534 | 0.444733 | 15.14236 | lung | non-small cell carcinoma |
| GRANTA519 | 0.017643 | 0.01487 | 0.468567 | 17.90564 | haematopoietic | mantle cell lymphoma |
| | | | | | and lymphoid | |
| | | | | | tissue | |
| DU145 | 0.01772 | 0.016588 | 0.432367 | 18.16451 | prostate | NS |
| SNU398 | 0.017992 | 0.009627 | 0.49108 | 25.8616 | liver | hepatocellular carcinoma |
| HLF | 0.018027 | 0.011534 | 0.465709 | 18.24934 | liver | hepatocellular carcinoma |
| NCIH1623 | 0.018862 | 0.00813 | 0.522025 | 30.06498 | lung | adenocarcinoma |
| SNU668 | 0.01888 | 0.012849 | 0.464013 | 16.0231 | stomach | signet ring adenocarcinoma |
| BXPC3 | 0.019388 | 0.008453 | 0.57381 | 36.5952 | pancreas | ductal carcinoma |
| NCIH1930 | 0.020006 | 0.006616 | 0.540233 | 36.32772 | lung | small cell carcinoma |
| HCT116 | 0.020431 | 0.004991 | 0.535649 | 35.91847 | large intestine | NS |
| NCIH1355 | 0.020573 | 10 | 0.559364 | 35.37581 | lung | adenocarcinoma |
| T84 | 0.020979 | 0.010096 | 0.492753 | 21.54238 | large intestine | adenocarcinoma |
| DMS273 | 0.022219 | 0.016264 | 0.481488 | 16.57774 | lung | small cell carcinoma |
| ECGI10 | 0.02244 | 10 | 0.568334 | 39.22919 | oesophagus | NS |
| 786O | 0.022468 | 0.011656 | 0.504861 | 22.93315 | kidney | clear cell renal cell |
| | | | | | | carcinoma |
| HCC78 | 0.02392 | 0.012709 | 0.531509 | 23.40461 | lung | adenocarcinoma |
| NCIH2122 | 0.023938 | 0.016742 | 0.501905 | 18.69937 | lung | adenocarcinoma |
| OVISE | 0.023953 | 10 | 0.540484 | 41.60625 | ovary | clear cell carcinoma |
| LUDLU1 | 0.025828 | 0.012626 | 0.530668 | 28.03128 | lung | squamous cell carcinoma |
| GP2D | 0.026231 | 0.020627 | 0.506015 | 20.31771 | large intestine | adenocarcinoma |
| WM2664 | 0.027533 | 10 | 0.583762 | 37.49186 | skin | NS |
| PLCPRF5 | 0.02796 | 0.0164 | 0.510666 | 21.67484 | liver | hepatocellular carcinoma |
| SW620 | 0.02926 | 0.026311 | 0.465758 | 9.079409 | large intestine | adenocarcinoma |
| SKCO1 | 0.030585 | 10 | 0.595243 | 34.7288 | large intestine | adenocarcinoma |
| A549 | 0.032375 | 0.018492 | 0.573044 | 31.10718 | lung | non-small cell carcinoma |
| NCIH929 | 0.034475 | 0.020579 | 0.52854 | 24.32478 | haematopoietic | plasma cell myeloma |
| | | | | | and lymphoid | |
| | | | | | tissue | |
| ACHN | 0.034708 | 0.030334 | 0.453014 | 2.196602 | kidney | renal cell carcinoma |
| NCIH1975 | 0.034766 | 0.025525 | 0.528991 | 17.89159 | lung | non-small cell carcinoma |
| PANC0213 | 0.035795 | 0.021461 | 0.582932 | 32.945 | pancreas | NS |
| HT1376 | 0.036268 | 0.017852 | 0.551857 | 18.92436 | urinary tract | transitional cell carcinoma |
| HT29 | 0.037031 | 0.022837 | 0.574146 | 29.83252 | large intestine | adenocarcinoma |
| UMUC3 | 0.039504 | 0.021864 | 0.593428 | 35.04699 | urinary tract | transitional cell carcinoma |
| SNU761 | 0.039689 | 10 | 0.581426 | 38.06756 | liver | hepatocellular carcinoma |
| SKLU1 | 0.040003 | 0.008488 | 0.56624 | 38.28472 | lung | adenocarcinoma |
| HCC1954 | 0.040563 | 10 | 0.591175 | 43.62567 | breast | ductal carcinoma |
| NCIH23 | 0.042711 | 0.037736 | 0.622699 | 27.39399 | lung | non-small cell carcinoma |
| KATOIII | 0.043772 | 0.040387 | 0.490001 | 12.87902 | stomach | adenocarcinoma |
| A2058 | 0.046764 | 0.028087 | 0.56942 | 30.63873 | skin | NS |
| SKMES1 | 0.047461 | 0.024323 | 0.60692 | 30.65752 | lung | squamous cell carcinoma |
| SW48 | 0.050006 | 10 | 0.603764 | 37.9499 | large intestine | adenocarcinoma |
| HEPG2 | 0.052079 | 10 | 0.608647 | 37.75892 | liver | hepatocellular carcinoma |
| SNUC2A | 0.052512 | 0.024542 | 0.615038 | 34.41705 | large intestine | adenocarcinoma |
| LOVO | 0.057826 | 10 | 0.636191 | 47.95459 | large intestine | adenocarcinoma |
| HSC3 | 0.060326 | 10 | 0.6313 | 47.25644 | upper | squamous cell carcinoma |
| | | | | | aerodigestive tract | |
| JIMT1 | 0.061923 | 0.046579 | 0.551921 | 13.45549 | breast | ductal carcinoma |
| SW1710 | 0.063291 | 0.027254 | 0.5768 | 25.48455 | urinary tract | transitional cell carcinoma |
| RERFLCMS | 0.065898 | 0.023477 | 0.641498 | 38.15621 | lung | non-small cell carcinoma |
| HS294T | 0.066791 | 0.030717 | 0.600533 | 28.13572 | skin | NS |
| ASPC1 | 0.066825 | 0.018855 | 0.581226 | 32.80606 | pancreas | ductal carcinoma |
| PANC1 | 0.06858 | 0.051468 | 0.554273 | 12.02616 | pancreas | ductal carcinoma |
| CAPAN1 | 0.115609 | 10 | 0.639142 | 40.2242 | pancreas | ductal carcinoma |
| NCIH446 | 0.119463 | 0.014536 | 0.583736 | 24.17448 | lung | small cell carcinoma |
| HEP3B217 | 0.129298 | 10 | 0.644762 | 47.20301 | liver | hepatocellular carcinoma |
| NCIH2172 | 0.132833 | 10 | 0.68115 | 37.97151 | lung | non-small cell carcinoma |
| SKMEL2 | 0.13968 | 0.026999 | 0.639177 | 35.37217 | skin | NS |
| SNU5 | 0.146396 | 0.082367 | 0.683652 | 32.15273 | stomach | undifferentiated |
| | | | | | | adenocarcinoma |
| NCIH2228 | 0.178529 | 10 | 0.686403 | 44.26922 | lung | adenocarcinoma |
| BICR18 | 0.19427 | 0.186872 | 0.618995 | 4.76155 | upper | squamous cell carcinoma |
| GI1 | 0.28744 | 0.295062 | 0.623711 | 9.190768 | aerodigestive tract | gliosarcoma |
| | | | | | central nervous | |
| | | | | | system | |
| MCF7 | 0.434714 | 10 | 0.659214 | 43.69748 | breast | NS |
| SKMEL3 | 0.472006 | 10 | 0.620722 | 46.89633 | skin | NS |
| WM115 | 0.477738 | 0.002502 | 0.609719 | 36.92872 | skin | NS |
| COLO679 | 2.044655 | 2.344991 | 0.502041 | 24.71872 | skin | NS |
| SNGM | 4.901703 | 4.138108 | 0.916457 | 26.87739 | endometrium | adenocarcinoma |
| NCIH2171 | 10 | 10 | 0.7416 | 16.52996 | lung | small cell carcinoma |
| SUPT1 | 3.3333 | 10 | 0.691296 | 48.11215 | haematopoietic | acute lymphoblastic T cell |
| | | | | | and lymphoid | leukaemia |
| | | | | | tissue | |
| KNS81 | 10 | 10 | 0.630939 | 58.5801 | central nervous | astrocytoma Grade IV |
| | | | | | system | |
| MPP89 | 10 | 10 | 0.638426 | 51.42635 | pleura | NS |
| SNU886 | 10 | 10 | 0.642533 | 48.34561 | liver | hepatocellular carcinoma |
| COLO829 | 10 | 10 | 0.655706 | 47.96395 | skin | NS |
| KALS1 | 10 | 10 | 0.65593 | 49.78685 | central nervous | NS |
| | | | | | system | |
| SNU878 | 10 | 10 | 0.664801 | 52.14968 | liver | hepatocellular carcinoma |
| GAK | 10 | 10 | 0.664855 | 47.69716 | NOT IN CCLE | NOT IN CCLE |
| OSRC2 | 10 | 10 | 0.665792 | 52.3524 | kidney | renal cell carcinoma |
| RCM1 | 10 | 10 | 0.671054 | 42.54585 | large intestine | adenocarcinoma |
| NCIH1734 | 10 | 10 | 0.673415 | 54.90475 | lung | adenocarcinoma |
| DMS153 | 10 | 10 | 0.673713 | 50.81473 | lung | small cell carcinoma |
| J82 | 10 | 10 | 0.674852 | 48.70006 | urinary tract | transitional cell carcinoma |
| BICR22 | 10 | 10 | 0.682919 | 54.75925 | upper | squamous cell carcinoma |
| | | | | | aerodigestive tract | |
| SW1990 | 10 | 10 | 0.689389 | 49.22702 | pancreas | ductal carcinoma |
| HCC1143 | 10 | 10 | 0.69059 | 50.38194 | breast | ductal carcinoma |
| HCC2935 | 10 | 10 | 0.692215 | 50.08429 | lung | non-small cell carcinoma |
| KYSE70 | 10 | 10 | 0.694176 | 50.18949 | oesophagus | squamous cell carcinoma |
| MDAMB468 | 10 | 10 | 0.697183 | 50.86583 | breast | NS |
| NMCG1 | 10 | 10 | 0.701293 | 47.05987 | central nervous | NS |
| | | | | | system | |
| SKHEP1 | 10 | 10 | 0.70906 | 44.3029 | liver | adenocarcinoma |
| SW837 | 10 | 10 | 0.710855 | 46.73384 | large intestine | adenocarcinoma |
| A204 | 10 | 10 | 0.713626 | 58.90593 | soft tissue | NS |
| HCC1419 | 10 | 10 | 0.720097 | 52.41521 | breast | ductal carcinoma |
| BT474 | 10 | 10 | 0.720573 | 53.87865 | breast | ductal carcinoma |
| MEWO | 10 | 10 | 0.723674 | 56.56317 | skin | NS |
| MDAMB231 | 10 | 10 | 0.726163 | 55.94067 | breast | NS |
| CALU3 | 10 | 10 | 0.727683 | 50.88262 | lung | adenocarcinoma |
| SKNSH | 10 | 10 | 0.729486 | 53.89154 | autonomic ganglia | NS |
| ONS76 | 10 | 10 | 0.73362 | 54.63371 | central nervous | NS |
| | | | | | system | |
| MDAMB 175 VII | 10 | 10 | 0.738455 | 60.66071 | breast | ductal carcinoma |
| SKMEL31 | 10 | 10 | 0.739322 | 59.48678 | skin | NS |
| DMS53 | 10 | 10 | 0.742606 | 49.07034 | lung | small cell carcinoma |
| SNUC1 | 10 | 10 | 0.747771 | 55.45382 | large intestine | adenocarcinoma |
| NCIH1573 | 10 | 10 | 0.748221 | 53.57475 | lung | adenocarcinoma |
| NCIH1435 | 10 | 10 | 0.761242 | 60.04075 | lung | non-small cell carcinoma |
| NIHOVCAR3 | 10 | 10 | 0.777131 | 59.47137 | ovary | NS |
| SCC25 | 10 | 10 | 0.778864 | 62.3079 | upper | squamous cell carcinoma |
| | | | | | aerodigestive tract | |
| NCIH441 | 10 | 10 | 0.78042 | 67.48763 | lung | adenocarcinoma |
| HCC1428 | 10 | 10 | 0.781839 | 59.9889 | breast | NS |
| VCAP | 10 | 10 | 0.782184 | 62.59825 | prostate | adenocarcinoma |
| NCIH1838 | 10 | 10 | 0.785788 | 60.0585 | lung | non-small cell carcinoma |
| SKMEL24 | 10 | 10 | 0.7882 | 58.11712 | skin | NS |
| LU99 | 10 | 10 | 0.78999 | 64.65551 | lung | large cell carcinoma |
| NCIH2009 | 10 | 8.019371 | 0.791426 | 55.69455 | lung | adenocarcinoma |
| RPMI7951 | 10 | 10 | 0.806669 | 66.30924 | skin | NS |
| NCIH596 | 10 | 10 | 0.815117 | 61.33612 | lung | mixed adenosquamous |
| | | | | | | carcinoma |
| TE8 | 10 | 10 | 0.816021 | 72.1307 | oesophagus | squamous cell carcinoma |
| SW1271 | 10 | 10 | 0.817932 | 65.37809 | lung | small cell carcinoma |
| C2BBE1 | 10 | 10 | 0.830329 | 59.34028 | large intestine | adenocarcinoma |
| CAL27 | 10 | 10 | 0.833497 | 74.67924 | upper | squamous cell carcinoma |
| | | | | | aerodigestive tract | |
| MDAMB157 | 10 | 10 | 0.836176 | 64.71391 | breast | ductal carcinoma |
| UACC812 | 10 | 6.947929 | 0.841822 | 62.67078 | breast | ductal carcinoma |
| MDAMB436 | 10 | 10 | 0.843786 | 70.42433 | breast | NS |
| NCIH1395 | 10 | 10 | 0.854187 | 70.68192 | lung | adenocarcinoma |
| HS739T | 10 | 10 | 0.868583 | 78.34986 | breast | NS |
| KURAMOCHI | 10 | 10 | 0.874218 | 73.84661 | ovary | undifferentiated carcinoma |
| NCIH1650 | 10 | 10 | 0.874566 | 68.07063 | lung | bronchioloalveolar |
| | | | | | | adenocarcinoma |
| CCFSTTG1 | 10 | 10 | 0.879034 | 78.89955 | central nervous | astrocytoma |
| | | | | | system | |
| CAPAN2 | 10 | 10 | 0.880831 | 75.01844 | pancreas | ductal carcinoma |
| HCC827 | 10 | 10 | 0.881358 | 74.84 | lung | adenocarcinoma |
| KASUMI1 | 10 | 10 | 0.882289 | 81.08059 | haematopoietic | acute myeloid leukaemia |
| | | | | | and lymphoid | |
| | | | | | tissue | |
| HS852T | 10 | 10 | 0.883001 | 78.51343 | skin | NS |
| HT144 | 10 | 10 | 0.89125 | 81.60806 | skin | NS |
| HCC1937 | 10 | 10 | 0.9094 | 80.9493 | breast | ductal carcinoma |
| BT20 | 10 | 10 | 0.911675 | 78.79204 | breast | ductal carcinoma |
| SW579 | 10 | 10 | 0.920274 | 79.98259 | thyroid | anaplastic carcinoma |
| HS695T | 10 | 10 | 0.923787 | 74.11092 | skin | NS |
| T24 | 10 | 10 | 0.928907 | 75.74279 | urinary tract | transitional cell carcinoma |
| TE1 | 10 | 10 | 0.937963 | 84.54246 | oesophagus | squamous cell carcinoma |
| MDAMB415 | 10 | 10 | 0.941323 | 83.39028 | breast | NS |
| SCC9 | 10 | 10 | 0.942441 | 69.24641 | upper | squamous cell carcinoma |
| | | | | | aerodigestive tract | |
| SCC15 | 10 | 10 | 0.948945 | 90.39119 | upper | squamous cell carcinoma |
| | | | | | aerodigestive tract | |
| KG1 | 10 | 10 | 0.959161 | 80.43661 | haematopoietic | acute myeloid leukaemia |
| | | | | | and lymphoid | |
| | | | | | tissue | |
| SW1417 | 10 | 10 | 0.963372 | 91.96064 | large intestine | adenocarcinoma |
| NCIH1563 | 10 | 10 | 0.965176 | 93.507 | lung | adenocarcinoma |
| DAOY | 10 | 10 | 0.968421 | 83.40166 | central nervous | NS |
| | | | | | system | |
| HUH28 | 10 | 10 | 0.98625 | 92.15381 | biliary tract | NS |
| HCC38 | 10 | 10 | 0.98639 | 93.61212 | breast | ductal carcinoma |
| MALME3M | 10 | 10 | 0.986698 | 94.08447 | skin | NS |
| SKOV3 | 10 | 10 | 0.990095 | 96.03318 | ovary | adenocarcinoma |
| G361 | 10 | 10 | 1.043507 | 89.52446 | skin | NS |
| SAOS2 | 10 | 10 | 1.080519 | 92.52133 | bone | NS |
| TE15 | 10 | 10 | 1.084416 | 89.04183 | oesophagus | squamous cell carcinoma |
| HS839T | 10 | 10 | 1.09737 | 101.2434 | skin | NS |
| HS688AT | 10 | 10 | 1.105351 | 98.46712 | skin | NS |
| MDAMB361 | 10 | 10 | 1.116565 | 94.55241 | breast | NS |
| CALU1 | 10 | 10 | 1.217208 | 118.4694 | lung | squamous cell carcinoma |
| NCIH1755 | 10 | 10 | 1.289838 | 123.3293 | lung | adenocarcinoma |

### Example 3: Determination of Anti-Proliferative Activity in a Xenograph Model

The therapeutic in vivo efficacy of Compound 1 was evaluated in SU60 colorectal cancer, KYSE-150 esophageal cancer, COH70 triple-negative breast cancer, GXA-3036 gastric adenocarcinoma and OCI-LY19 Diffuse Large B-Cell Non-Hodgkin's Lymphoma xenograft models. In all studies, efficacy was determined based on percent tumor growth inhibition (TGI), differences in mean tumor growth, and statistical assessment of differences in net tumor volume distributions on the day of TGI analysis between control and treated animals. Tolerability was assessed based on differences in mean body weights between control and treated animals over the course of the study.

### A. In Vivo Efficacy of Compound 1 in the SU60 Patient-Derived Colorectal Cancer Xenograft Model

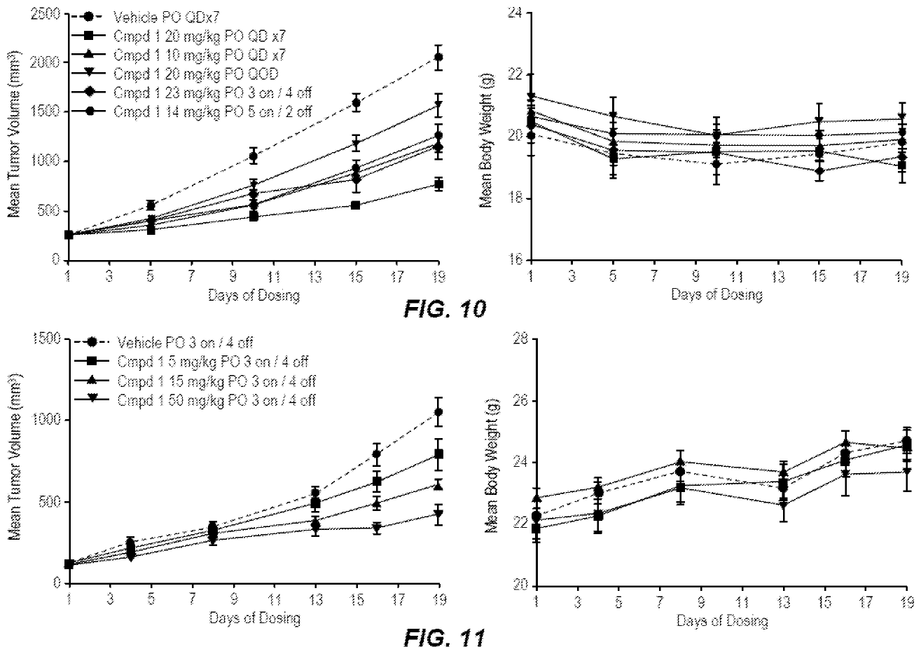

The in vivo therapeutic efficacy and tolerability of Compound 1, administered orally as the free acid form in a vehicle comprised of 50% polyethylene glycol 400 (PEG 400) and 50% PBS (pH 9), was evaluated in the SU60 CRC PDX model established in female non-obese diabetic/severe combined immunodeficiency (NOD/SCID) gamma (NSG) mice. Each mouse was inoculated subcutaneously in the abdomen with 2×106 SU60 tumor cells (0.1 ml) suspended in Matrigel. When the average tumor size reached ˜270 mm3, test animals were randomized into 6 groups consisting of 8 mice per group and treatments were initiated. Control mice received 50% PEG 400 and 50% PBS (pH 9) vehicle PO QD×7. Treated mice received Compound 1 in vehicle PO at 20 or 10 mg/kg QD×7, at 20 mg/kg QOD, at 23 mg/kg 3 on/4 off, or at 14 mg/kg at 5 on/2 off. TGI was determined on Day 19 as (1−Diff treated/Diff control)×100. The difference in mean net tumor volumes on the day of TGI analysis for treated versus control animals was evaluated statistically using one-way analysis of variance (ANOVA) followed by Dunnett's multiple comparisons test. Adjusted p-values are shown where a calculated probability (p)≤0.05 was considered statistically significant. Individual tumors were measured twice weekly in two dimensions using a caliper, and the tumor volumes (TV) in mm3 were calculated using the formula: TV=0.5 (l×w×h), where l, w, and h are the tumor length, width, and height in mm, respectively; Mean tumor volumes±standard error of the mean (SEM) were calculated and plotted versus days of dosing (FIG. 10 left panel). Body weights were measured twice weekly. Body weights±SEM were calculated and plotted versus days of dosing (FIG. 10 right panel).

As presented in Table 4, all five Compound 1 regimens were significantly efficacious (p<0.005) based on the statistical assessment of differences in mean net tumor volumes on the day of TGI analysis for treated versus control animals. Compound 1 at 20 or 10 mg/kg dosed once daily for 7 days (QD×7) yielded dose-dependent TGIs of 71% and 48%. Four Compound 1 dosing regimens: QD×7, once every other day (QOD), once daily for 3 days followed by no dosing for 4 days repeated to study end (3 on/4 off), and 5 on/2 off were tested at similar weekly dose levels (69 to 70 mg/kg/wk) to identify an optimal intermittent dosing schedule. The intermittent schedules of 3 on/4 off and 5 on/2 off delivered similar tumor growth responses as QD×7 (52% and 42% vs. 48% TGI). Compound 1 at 20 mg/kg administered QOD was the least efficacious regimen yielding 29% TGI.

As shown in FIG. 10 (left panel), tumor growth in the Compound 1-treated groups was reduced relative to control animals. The relative rates of tumor growth among the various Compound 1-treated groups were consistent with the TGI attained at study end. As presented in FIG. 10 (right panel), Compound 1 appeared well-tolerated with nominal mean body weight changes over the course of the study.

TABLE 4

| Treatment Regimen | | | | | | | | | | Statistical |
|---|---|---|---|---|---|---|---|---|---|---|
| | | Dose (mg/kg) | | | | Median Tumor Volumes (mm³) | | | | Significance |
| n | Agent | Single | Weekly | Route | Schedule | Day 1 | Day 19 | Diff. | % TGI | (p-value) |
| 8 | Vehicle | — | — | PO | QD x7 | 266 | 2011 | 1745 | — | — |
| 8 | Compd 1 | 20 | 140 | PO | QD x7 | 261 | 767 | 506 | 71 | <0.0001 |
| 8 | Compd 1 | 10 | 70 | PO | QD x7 | 250 | 1151 | 901 | 48 | <0.0001 |
| 8 | Compd 1 | 20 | 70 | PO | QOD | 285 | 1527 | 1242 | 29 | 0.0049 |
| 8 | Compd 1 | 23 | 69 | PO | 3 on/4 off | 288 | 1121 | 833 | 52 | <0.0001 |
| 8 | Compd 1 | 14 | 70 | PO | 5 on/2 off | 281 | 1300 | 1019 | 42 | <0.0001 |

### B. In Vivo Efficacy of Compound 1 in the KYSE-150 Human Esophageal Cancer Cell Line Xenograft Model

Study #1—The in vivo therapeutic efficacy and tolerability of Compound 1, administered orally as the lysine salt suspended in a vehicle comprised of 10% PEG 400 and 90% of 0.5% methylcellulose (MC), was evaluated in the KYSE-150 ESCC CDX model established in female NSG mice. As presented in Table 5, compound 4 at 5, 15, or 50 mg/kg (free acid equivalents) administered on the 3 on/4 off schedule yielded dose-dependent TGIs of 19%, 50%, and 72%, respectively. Differences in mean net tumor volumes on the day of TGI analysis for treated versus control animals were significant for Compound 1 at 15 and 50 mg/kg (p<0.001) but not at 5 mg/kg (p>0.05). As shown in FIG. 11 (left panel), tumor growth in the Compound 1-treated groups was reduced relative to control animals. The relative rates of tumor growth among the various Compound 1-treated groups were consistent with the TGI attained at study end. As presented in FIG. 11 (right panel), Compound 1 appeared well-tolerated with nominal mean body weight gains over the course of the study. Each mouse was inoculated subcutaneously in the right flank with 1×107 KYSE-150 tumor cells (0.1 ml) suspended in PBS. When the average tumor size reached ˜117 mm3, test animals were randomized into 4 groups consisting of 9 mice per group and treatments were initiated. Control mice received 10% PEG 400 and 90% of 0.5% MC vehicle PO 3 on/4 off. Treated mice received the Compound 1 lysine salt form suspended in vehicle at 5, 15, or 50 mg/kg on the same schedule. TGI was determined on Day 19 as (1−Difftreated/Diffcontrol)×100; The difference mean net tumor volumes on the day of TGI analysis for treated versus control animals was evaluated statistically using one-way ANOVA followed by Dunnett's multiple comparisons test; Adjusted p-values are shown where a calculated probability (p)≤0.05 was considered statistically significant. Individual tumors were measured twice weekly in three dimensions using a caliper, and the tumor volumes (TV) in mm3 were calculated using the formula: TV=0.5 (l×w×h), where l, w, and h are the tumor length, width, and height in mm, respectively; Mean tumor volumes SEM were calculated and plotted versus days of dosing (left panel). Body weights were measured twice weekly. Body weights±SEM were calculated and plotted versus days of dosing (right panel).

TABLE 5

| | Treatment Regimen | | | | | | | | Statistical |
|---|---|---|---|---|---|---|---|---|---|
| | | Dose | | | Median Tumor Volumes (mm³) | | | | Significance |
| n | Agent | (mg/kg) | Route | Schedule | Day 1 | Day 19 | Diff. | % TGI | (p-value) |
| 9 | Vehicle | — | PO | 3 on/4 off | 104 | 988 | 884 | — | — |
| 9 | Compound 1 | 5 | PO | 3 on/4 off | 105 | 824 | 719 | 19% | 0.0538 |
| 9 | Compound 1 | 15 | PO | 3 on/4 off | 127 | 569 | 442 | 50% | 0.0006 |
| 9 | Compound 1 | 50 | PO | 3 on/4 off | 109 | 359 | 250 | 72% | <0.0001 |

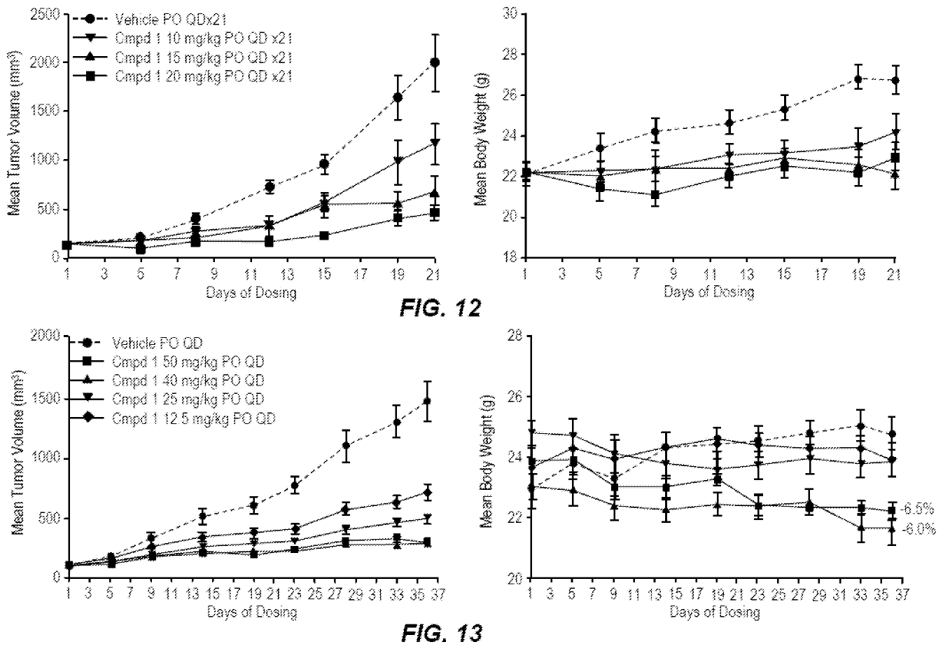

Study #2—The in vivo therapeutic efficacy and tolerability of Compound 1, administered orally as the lysine salt suspended in a vehicle comprised of 10% PEG 400 and 90% of 0.5% MC, was evaluated in the KYSE-150 ESCC CDX model established in female NOD/SCID mice. Each mouse was inoculated subcutaneously in the right flank with 1×107 KYSE-150 tumor cells (0.1 ml) suspended in 50% Matrigel. When the average tumor size reached ˜153 mm3, test animals were randomized into 4 groups consisting of 8 mice per group and treatments were initiated. Control mice received 10% PEG 400 and 90% of 0.5% MC vehicle PO QDx21. Treated mice received the Compound 1 lysine salt form suspended in vehicle at 10, 15, or 20 mg/kg on the same schedule. TGI was determined on Day 21 as (1−Difftreated/Diffcontrol)×100. Individual tumors were measured twice weekly in two dimensions using a caliper, and the tumor volumes (TV) in mm3 were calculated using the formula: TV=0.5 a×b2, where a and b are the long and short tumor diameters in mm, respectively; Mean tumor volumes±SEM were calculated and plotted versus days of dosing (FIG. 12 left panel). Body weights were measured twice weekly. Body weights±SEM were calculated and plotted versus days of dosing (FIG. 12 right panel).

The difference in mean net tumor volumes on the day of TGI analysis for treated versus control animals was evaluated statistically using one-way ANOVA followed by Dunnetts multiple comparisons test. Adjusted p-values are shown where a calculated probability (p)≤0.05 was considered statistically significant.

As presented in Table 6, Compound 1 at 10, 15, or 20 mg/kg (free acid equivalents) administered on the QD×21 schedule yielded dose-dependent TGIs of 54%, 76%, and 84%, respectively. Differences in mean net tumor volumes on the day of TGI analysis for treated versus control animals were significant (p<0.02).

As shown in FIG. 12 (left panel), tumor growth in the Compound 1-treated groups was reduced relative to control animals. The relative rates of tumor growth among the various Compound 1-treated groups were consistent with the TGI attained at study end. As presented in FIG. 12 (right panel), Compound 1-treated animals did not experience the body weight gains exhibited by control animals but appeared well-tolerated with nominal mean body weight changes over the course of the study.

TABLE 6

| | Compound Treatment Regimen | | | | | | | | Statistical |
|---|---|---|---|---|---|---|---|---|---|
| | | Dose | | | Median Tumor Volumes (mm³) | | | | Significance |
| n | Agent | (mg/kg) | Route | Schedule | Day 1 | Day 21 | Diff. | % TGI | (p-value) |
| 8 | Vehicle | — | PO | QD x21 | 153 | 2076 | 1923 | — | — |
| 8 | Compound 1 | 10 | PO | QD x21 | 151 | 1027 | 876 | 54 | 0.0155 |
| 8 | Compound 1 | 15 | PO | QD x21 | 152 | 620 | 468 | 76 | 0.0002 |
| 8 | Compound 1 | 20 | PO | QD x21 | 154 | 469 | 315 | 84 | <0.0001 |

### C. In Vivo Efficacy of Compound 1 in the COH70 Patient-Derived Triple Negative Breast Cancer Xenograft Model

The in vivo therapeutic efficacy and tolerability of Compound 1, administered orally as the free acid form in a PBS vehicle plus two equivalents of NaOH (final pH 10), was evaluated in a dose range study using the COH70 TNBC PDX model established in female NSG mice. Each mouse was inoculated subcutaneously in the abdomen with 2×106 COH70 tumor cells (0.2 ml) suspended in Matrigel. When the average tumor size reached ˜107 mm3, test animals were randomized into 5 groups consisting of 9 mice per group and treatments were initiated. Control mice received PBS (pH 10) vehicle PO QDx36. Treated mice received Compound 1 (dissolved in PBS+2 equivalents of NaOH, final pH 10) PO at 50, 40, 25, or 12.5 mg/kg QDx36. TGI was determined on Day 36 as (1−Difftreated/Diffcontrol)×100. Individual tumors were measured twice weekly in two dimensions using a caliper, and the tumor volumes (TV) in mm3 were calculated using the formula: TV=0.5 (l×w×h), where l, w, and h are the tumor length, width, and height in mm, respectively. Mean tumor volumes±SEM were calculated and plotted versus days of dosing (left panel). Body weights were measured twice weekly. Body weights±SEM were calculated and plotted versus days of dosing (right panel).

The difference in the distribution of net tumor volumes on the day of TGI analysis for treated versus control animals was evaluated statistically using one-way ANOVA followed by Dunnett's multiple comparisons test; Adjusted p-values are shown where a calculated probability (p)≤0.05 was considered statistically significant; These are tests of statistical significance and do not provide an estimate of the size of the difference between groups nor are they a measure of clinical or biological significance.

As presented in Table 7, Compound 1 at 12.5, 20, 40, or 50 mg/kg administered on the QD×36 schedule was significantly efficacious (p<0.0001), based on differences in mean net tumor volumes on the day of TGI analysis for treated versus control animal s, yielding respective TGIs of 57%, 68%, 86%, and 86%. Compound 1 yielded dose-dependent inhibition of tumor growth that attained a plateau at doses≥40 mg/kg.

As shown in FIG. 13 (left panel), tumor growth in the Compound 1-treated groups was reduced relative to control animals. The relative rates of tumor growth among the various Compound 1-treated groups were consistent with the TGI attained at study end. As presented in FIG. 13 (right panel), Compound 1 appeared well-tolerated with nominal dose-dependent mean body weight changes (6.5% mean body weight loss) over the course of the study.

TABLE 7

| Treatment Regimen | | | | | | | | | Statistical |
|---|---|---|---|---|---|---|---|---|---|
| | | Dose | | | Median Tumor Volumes (mm³) | | | | Significance |
| n | Agent | (mg/kg) | Route | Schedule | Day 1 | Day 36 | Diff. | % TGI | (p-value) |
| 9 | Vehicle | — | PO | QD x36 | 110 | 1455 | 1346 | — | — |
| 9 | Compound 1 | 12.5 | PO | QD x36 | 104 | 685 | 581 | 57 | <0.0001 |
| 9 | Compound 1 | 25 | PO | QD x36 | 99 | 524 | 424 | 68 | <0.0001 |
| 9 | Compound 1 | 40 | PO | QD x36 | 98 | 282 | 184 | 86 | <0.0001 |
| 9 | Compound 1 | 50 | PO | QD x36 | 103 | 295 | 191 | 86 | <0.0001 |

### D. In Vivo Efficacy of Compound 1 in the GXA-3036 Gastric Adenocarcinoma Patient-Derived Xenograft Model

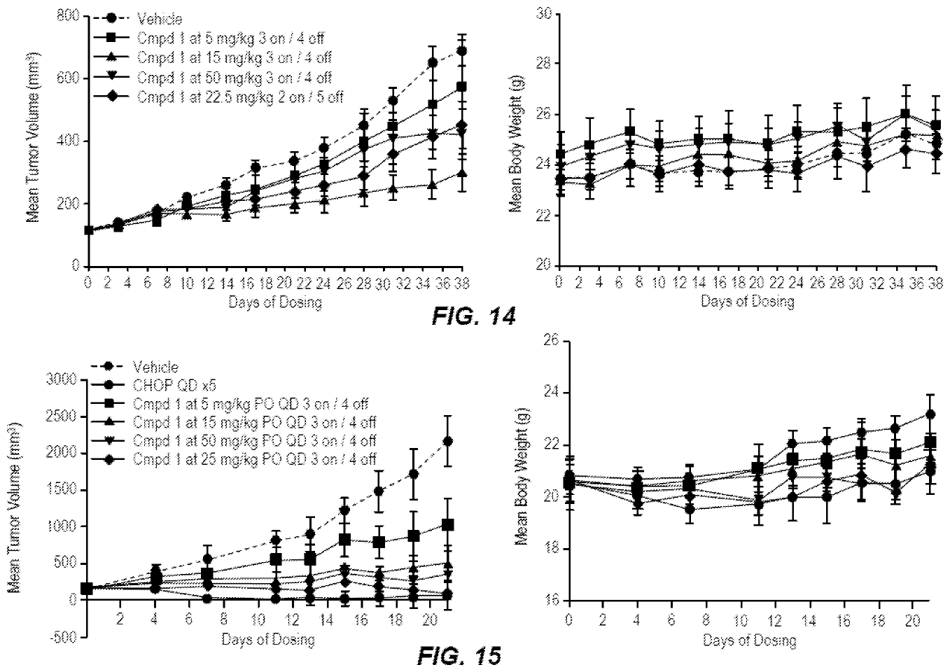

The in vivo efficacy and tolerability of Compound 1, administered orally as the lysine salt form suspended in a vehicle comprised of 5% 2-hydroxypropyl-beta-cyclodextrin (HPBCD) in 50 mM phosphate buffer (pH 7.4), were evaluated in female immunodeficient NMRI-Foxn1nu mice using the subcutaneous gastric adenocarcinoma GXA-3036, a moderately differentiated intestinal type (based on Lauren's criteria) PDX model of Asian origin. Each mouse was inoculated subcutaneously in the right flank with a GXA-3036 tumor fragment (3 to 4 mm edge length). When the average tumor size reached ˜109 mm3, test animals were randomized into 5 groups consisting of 8 mice per group and treatments were initiated. Control mice received 5% HPBCD in 50 mM phosphate buffer (pH 7.4) vehicle PO 3 on/4 off. Treated mice received the Compound 1 lysine salt form in vehicle at 5, 15, or 50 mg/kg on the same schedule or at 22.5 mg/kg 2 on/5 off. One control animal, classified as moribund, was euthanized on Day 17 and was censored from analysis. Individual tumors were measured twice weekly in two dimensions using a caliper, and the tumor volumes (TV) in mm3 were calculated using the formula: TV=0.5 a×b2, where a and b are the long and short diameters in mm, respectively. Mean tumor volumes±SEM were calculated and plotted versus days of dosing (FIG. 14 left panel). Body weights were measured twice weekly. Body weights±SEM were calculated and plotted versus days of dosing (FIG. 14 right panel).

TGI was determined on Days 35 and 38 as (1−Difftreated/Diffcontrol)×100. The differences in median net tumor volumes on the days of TGI analysis for treated versus control animals were evaluated statistically using the non-parametric Kruskal-Wallis test followed by Dunn's multiple comparisons test. The reported adjusted p-values are for both days of TGI analysis where a calculated probability (p)≤0.05 was considered statistically significant.

As presented in Table 8, Compound 1 at 5, 15, or 50 mg/kg (free acid equivalents) administered on the 3 on/4 off schedule yielded respective TGIs of 52%, 71%, and 55% on Day 35 and 43%, 69%, and 52% on Day 38. The lower TGI exhibited by the 5 mg/kg Compound 1 group on D38 was due to a moderate increase in tumor volume for a single animal that shifted the median. Compound 1 at 22.5 mg/kg (free acid equivalents) administered on the 2 on/5 off schedule yielded respective Day 35 and Day 38 TGIs of 42% and 41%. Differences in median net tumor volumes on both days of TGI analyses for treated versus control animals were significant for Compound 1 at 15 mg/kg (p=0.0007) but not for the other treatment regimens (p>0.05).

As shown in FIG. 14 (left panel), tumor growth in the Compound 1-treated groups was reduced relative to control animals. The relative rates of tumor growth among the various Compound 1-treated groups were consistent with the TGI attained at study end. As presented in FIG. 14 (right panel), Compound 1 appeared well-tolerated with little change in mean body weights over the course of the study.

TABLE 8

| | | Treatment Regimen | | | | | | | | | | | Statistical |
|---|---|---|---|---|---|---|---|---|---|---|---|---|---|
| | | | Dose | | | Median Tumor Volume (mm³) | | | | | | | Significance |
| Group | n | Agent | (mg/kg) | Route | Schedule | Day 0 | Day 35 | Diff. | % TGI | Day 38 | Diff. | % TGI | (p-value) |
| 1 | 7 | Vehicle | — | PO | 3 on/4 off | 111 | 698 | 587 | — | 728 | 617 | — | — |
| 2 | 8 | Compound 1 | 5 | PO | 3 on/4 off | 109 | 391 | 282 | 52% | 460 | 351 | 43% | >0.05 |
| 3 | 8 | Compound 1 | 15 | PO | 3 on/4 off | 109 | 278 | 169 | 71% | 298 | 189 | 69% | 0.0007 |
| 4 | 8 | Compound 1 | 50 | PO | 3 on/4 off | 109 | 371 | 262 | 55% | 403 | 294 | 52% | >0.05 |
| 5 | 8 | Compound 1 | 22.5 | PO | *2 on/5 off | 109 | 447 | 338 | 42% | 470 | 361 | 41% | >0.05 |

### E. In Vivo Efficacy of Compound 1 in the OCI-LY19 Human Diffuse Large B-Cell Non-Hodgkin's Lymphoma Xenograft Model

The in vivo efficacy and tolerability of Compound 1, administered orally as the lysine salt form suspended in a 0.5% MC vehicle, were evaluated in female C.B-17 severe combined immunodeficient (scid) mice inoculated subcutaneously with OCI-LY19 tumor cells established from the bone marrow of a female patient with diffuse large B-cell non-Hodgkin's lymphoma (DLBCL), stage 4B, at relapse. Each mouse was inoculated subcutaneously in the right flank with 5×106 OCI-LY19 tumor cells (0.1 ml) suspended in 50% Matrigel. When the average tumor size reached ˜165 mm3, test animals were randomized into 6 groups consisting of 8 mice per group and treatments were initiated. Study control mice received 0.5% MC vehicle PO QD 3 on/4 off and positive control mice received CHOP therapy QDx5. Compound 1-treated mice received the Compound 1 lysine salt form suspended in vehicle at 5, 15, or 50 mg/kg QD or at 25 mg/kg BID on the 3 on/4 off schedule. TGI was determined on Day 21 as (1−Difftreated/Diffcontrol)×100. Individual tumors were measured on the days shown in two dimensions using a caliper, and the tumor volumes (TV) in mm3 were calculated using the formula: TV=0.5 a×b2, where a and b are the long and short tumor diameters in mm, respectively. Mean tumor volumes±SEM were calculated and plotted versus days of dosing (FIG. 15 left panel). Body weights were measured twice weekly. Body weights±SEM were calculated and plotted versus days of dosing (FIG. 15 right panel). The difference in mean net tumor volumes on the day of TGI analysis for treated versus control animals was evaluated statistically using one-way ANOVA followed by Dunnett's multiple comparisons test; Adjusted p-values are shown where a calculated probability (p)≤0.05 was considered statistically significant.

As presented in Table 9, Compound 1 at 5, 15, or 50 mg/kg administered QD or at 25 mg/kg (free acid equivalents) administered twice daily (BID) on the 3 on/4 off schedule yielded respective dose-dependent TGIs of 55%, 83%, 90%, and 102% on Day 21. As shown in FIG. 15, differences in mean net tumor volumes on the day of TGI analysis for Compound 1-treated versus control animals were significant (p<0.0001).

The gold standard first line chemotherapy regimen CHOP (cyclophosphamide, doxorubicin, vincristine, and prednisone) recommended in the treatment of non-Hodgkin's lymphoma was included as a positive control. CHOP administered QD×5 yielded a TGI of 108% on Day 21 and appeared curative in seven of eight test animals that exhibited no measurable tumor volume for eight or more consecutive days.

As shown in FIG. 15 (left panel), tumor growth in the Compound 1-treated groups was reduced relative to control animals. The relative rates of tumor growth among the various Compound 1-treated groups were consistent with the TGI attained at study end. As presented in FIG. 15 (right panel), compound 1 appeared well-tolerated with little change in mean body weights over the course of the study.

TABLE 9

| | | Treatment Regimen | | | | | | | | Statistical |
|---|---|---|---|---|---|---|---|---|---|---|
| | | | Dose | | | Median Tumor Volume (mm³) | | | | Significance |
| Group | n | Agent | (mg/kg) | Route | Schedule | Day 0 | Day 21 | Diff. | % TGI | (p-value) |
| 1 | 8 | Vehicle | — | PO | QD 3 on/4 off | 167 | 2227 | 2061 | — | — |
| 2 | 8 | cyclophosphamide | 5 | IV | QD x5 | 164 | 0 | −164 | 108% | <0.0001 |
| | | doxorubicin | 0.5 | IV | | | | | | |
| | | vincristine | 0.08 | IV | | | | | | |
| | | prednisolone | 0.03 | PO | | | | | | |
| 3 | 8 | Compound 1 | 5 | PO | QD 3 on/4 off | 168 | 1088 | 920 | 55% | <0.0001 |
| 4 | 8 | Compound 1 | 15 | PO | QD 3 on/4 off | 167 | 512 | 345 | 83% | <0.0001 |
| 5 | 8 | Compound 1 | 50 | PO | QD 3 on/4 off | 165 | 361 | 196 | 90% | <0.0001 |
| 6 | 8 | Compound 1 | 25 | PO | BID 3 on/4 off | 167 | 135 | −32 | 102% | <0.0001 |

### Example 4: Determination of Cancer Stem Cell Characteristics Following Treatment with Compound 1

The ability of Compound 1 to affect tumorigenic potential was analyzed by flow cytometry analysis of the tumorigenic cell population and functional in vivo tumorigenicity assay (limiting dilution assay).

To assess the effect of Compound 1 on tumorigenic cell population, Compound 1 was used to treat SU60 xenograft tumors. NSG female mice engrafted with SU60 cells (7 mice each group) were administered Compound 1 at 40 mg/kg PO QD×21. The day after the last dose, three tumors nearest to the mean size from each group were dissociated and the single cells were used to perform: 1) flow cytometry analysis of the tumorigenic cell population and 2) functional in vivo tumorigenicity assay (limiting dilution assay). Flow Cytometry Analysis of Tumorigenic Cell Population after Compound 1 Treatment

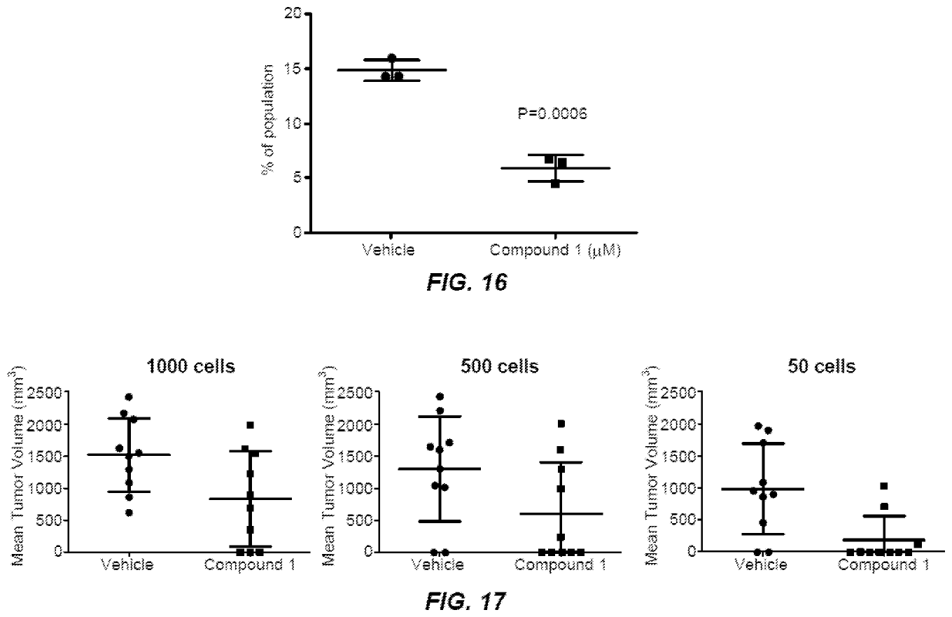

The proportion of tumorigenic CD44^{High};EpCAM⁺ cells in the cell population was quantified by flow cytometry. Compound 1 significantly reduced by 2.5 fold the proportion of tumorigenic cells (CD44^{High};EpCAM⁺) compared to vehicle control, see FIG. 16. Flow cytometry analysis performed on single cell isolated from 3 vehicle-treated and 3 Compound 1-treated tumors. P-value was determined with unpaired Student T-test.

Functional In Vivo Tumorigenicity Assay after Compound 1 Treatment

To study the effect of Compound 1 on tumor initiating cell (TIC) frequency in CRC tumors, we evaluated the tumorigenic potential of SU60 xenograft cells posttreatment with Compound 1 or vehicle. At the end of dosing, SU60 dissociated cells from three animals per group nearest to the mean size were injected in a limiting dilution manner into recipient NSG mice and tumor volumes were measured. The number of cells required for tumor formation was reduced in the Compound 1-treated group. The reduction in TIC frequency was 4.4-fold (FIG. 17 and Table 10). Differentiation of progenitor cells is a potential explanation for this reduction.

TABLE 10

| | Tumor | Tumor | Tumor | |
|---|---|---|---|---|
| | Take Rate | Take Rate | Take Rate | |
| | & Mean | & Mean | & Mean | |
| | Tumor | Tumor | Tumor | |
| Treatment | Volume | volume | Volume | TIC |
| Group | (1000 cells) | (500 cells) | (50 cells) | frequency |
| Vehicle | 10/10 | 8/10 | 8/10 | 1 in 144 |
| | 1588 mm³ | 1290 mm³ | 984 mm³ | cells |
| Compound 1, | 7/10 | 5/10 | 3/10 | 1 in 643 |
| 40 mg/kg, QD | 830 mm³ | 589 mm³ | 209 mm³ | cells |

### Example 5: Sensitivity of Various Colorectal Cancer Cells Lines with MSI-H Status to Compound 1 Treatment

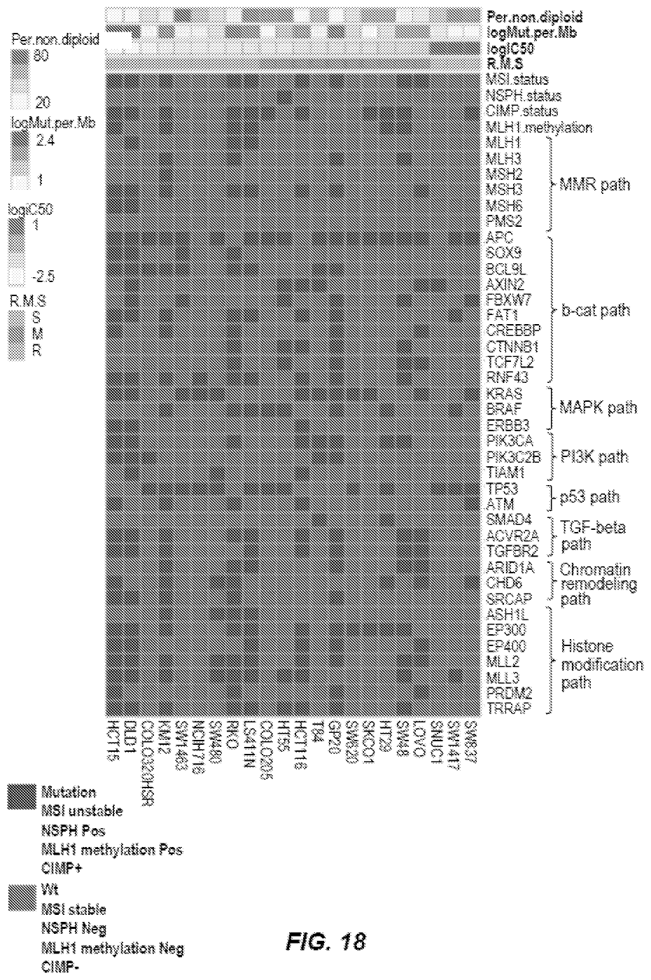

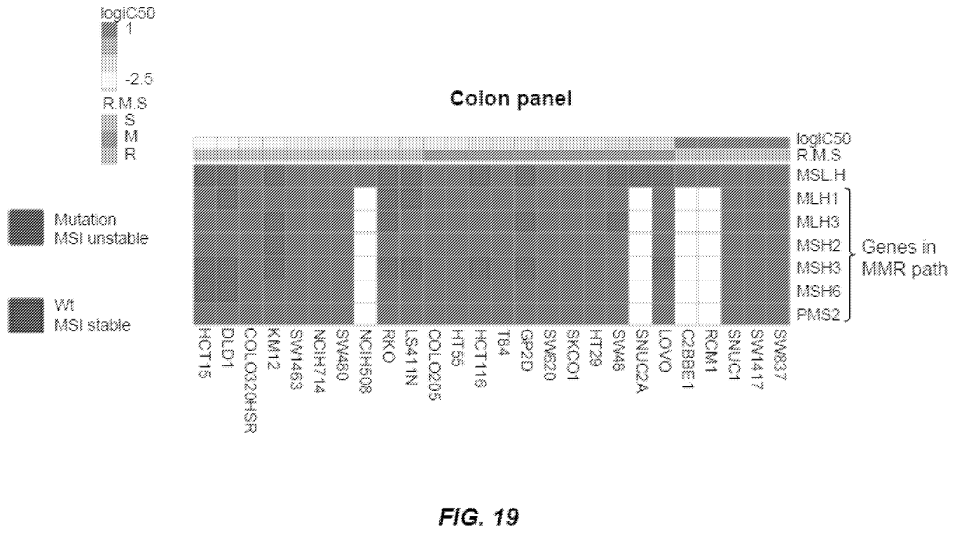

To further study the efficacy of Compound 1, the sensitivity of various colon cancer cell lines with certain genes of interest to Compound 1 was evaluated. These certain genes of interest are found in frequently mutated pathways such as gene mismatch repair, beta-catenin, MAPK, PI3K, p53, TGFbeta, chromatin remodeling, and histone modification pathways. FIG. 18 shows a heatmap of the mutation status of genes in these pathways along with the respective Microsatellite instability (MSI-H), CpG island methylator phenotype (CIMP), and MLH1 methylation status, in which the cell lines are sorted by their sensitivity to Compound 1.

Further studies showed that colorectal cancer cells lines with MSI-H status have significantly higher sensitivity to Compound 1 than those colorectal cancer cells lines that are microsatellite stable (MSS). A panel of 13 colorectal cancer PDX models and 6 colorectal cancer organoid PDX models were tested for their sensitivity to Compound 1 in culture-based viability inhibition assays. The MSI-H status of the studied colorectal cancer cell lines and their respective mutations of the MMR path genes are shown in FIG. 19.

As summarized in Table 11 below, it was found that 100% of the PDX models characterized as MSI (5 out of 5) were sensitive (IC₅₀<1 μM) to Compound 1 treatment and the IC₅₀'s ranged from 0.001 to 0.014 μM. However, only 50% of the PDX models characterized as MSS (4 out of 8) were sensitive to Compound 1 treatment and the IC₅₀'s ranged from 0.003 to 0.270 μM. Similarly, 100% of the organoid models characterized as MSI (3 out of 3) were sensitive to Compound 1 treatment (IC₅₀ ranged from 0.022 to 0.149 μM), while none of the organoid models characterized as MSS (0 out of 3) was sensitive to Compound 1 treatment (IC₅₀ values were greater than 10 μM).

TABLE 11

| | | |
|---|---|---|
| PDX Model Phenotype | Sensitive to Compound 1 | IC₅₀ (μM)^{a} |

| | | | |
|---|---|---|---|
| MSI | 5/5 | (100%) | 0.001-0.014 |
| MSI (Organoid) | 3/3 | (100%) | 0.022-0.149 |
| MSS | 4/8 | (50%) | 0.003-0.270 |
| MSS (Organoid) | 0/3 | (0%) | >10 |

Note:
MSI—Microsatellite instability;
MSS—Microsatellite stable
^{a}IC₅₀ shown only for those displaying sensitivity to Compound 1.

### Example 6: Identification of Biomarkers Associated with Compound 1 Treatment

RNA-seq and ChIP-seq analyses were employed to identify potential clinical pharmacodynamic (PD) biomarkers as molecular indicators for monitoring the effects of treatment with Compound 1.

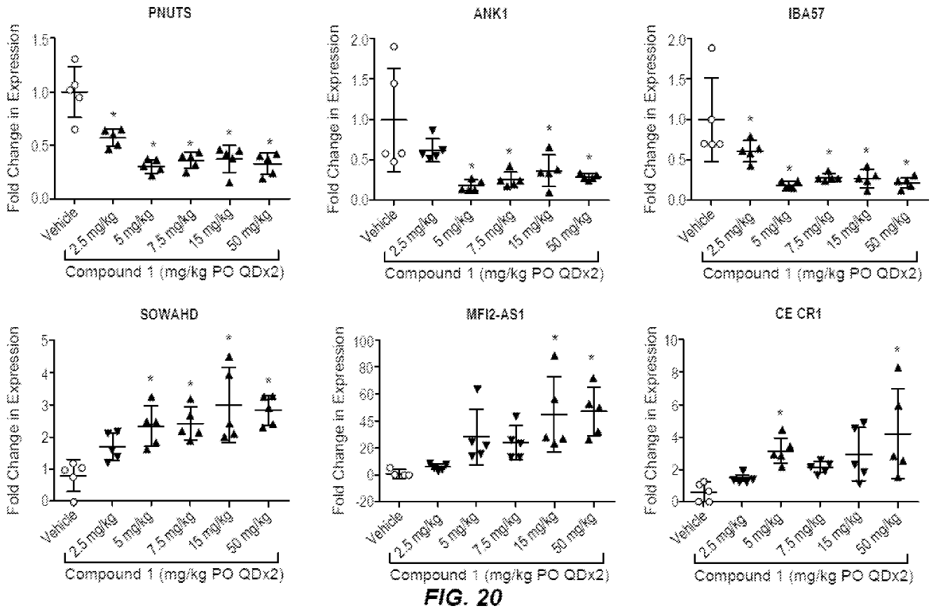

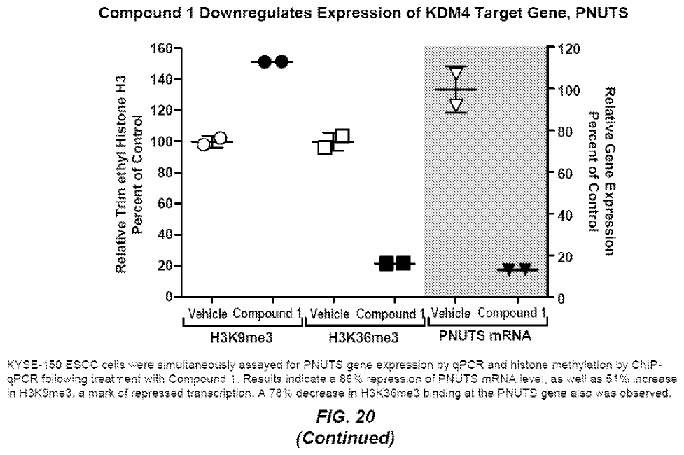

In the RNA-seq analysis performed, 326 genes that were up- or downregulated ≥2-fold by Compound 1 treatment were initially identified. The tested dose of Compound 1 was 37.3 mg/kg, which was the highest dose tested in diffuse large B-Cell Lymphoma xenografts models. In the ChIP-seq analysis, 65 genes were identified to show direct KDM4C promoter occupancy. FIG. 20 shows that genes PNUTS (PPP1R10), ANK1, IBA57, SOWAHD, MF12-AS1, and CECR1 exhibited dose-dependent changes following treatment with Compound 1 in vivo at 2.5 mg/kg, 5 mg/kg, 7.5 mg/kg, 15 mg/kg, and 50 mg/kg. FIG. 20 also shows that PNUTS, ANK1, and IBA57 are downregulated during Compound 1 treatment while SOWAHD, MF12-AS1, and CECR1 are upregulated.

Compound 1 treatment for 24 hours demonstrated potent inhibition of PNUTS gene expression. In a study of the inhibitory effects of Compound 1 on the gene expression of PNUTS, MDA-MB-231 cells were treated for 24 hours and expression levels of PNUTS mRNA in response to Compound 1 treatment were measured by qPCR. The inhibition of PNUTS mRNA was quantified by calculating mean percent change in PNUTS gene expression following the Compound 1 treatment relative to the DMSO-treated control and IC₅₀ value was calculated as concentration where inhibition was half-maximal. The IC₅₀ value was 0.007 μM.

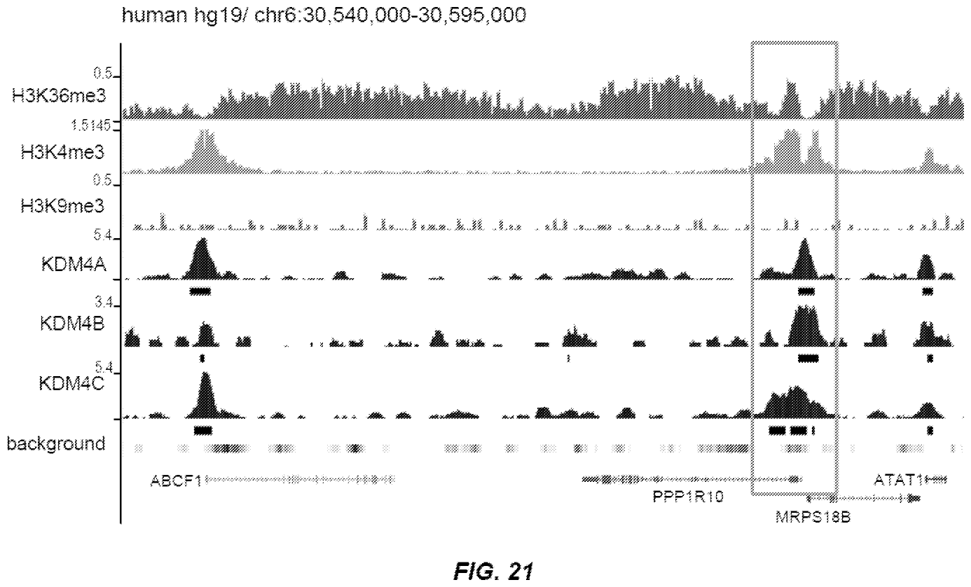

Moreover, ChIP-seq analysis in MDA-MB-231 cells shows KDM4 occupancy at the PNUTS (PPP1R10) promoter as marked by H3K4me3 (FIG. 21)

### Example 7: Single Cell Gene Expression Analysis of Immature and Mature Cell Markers

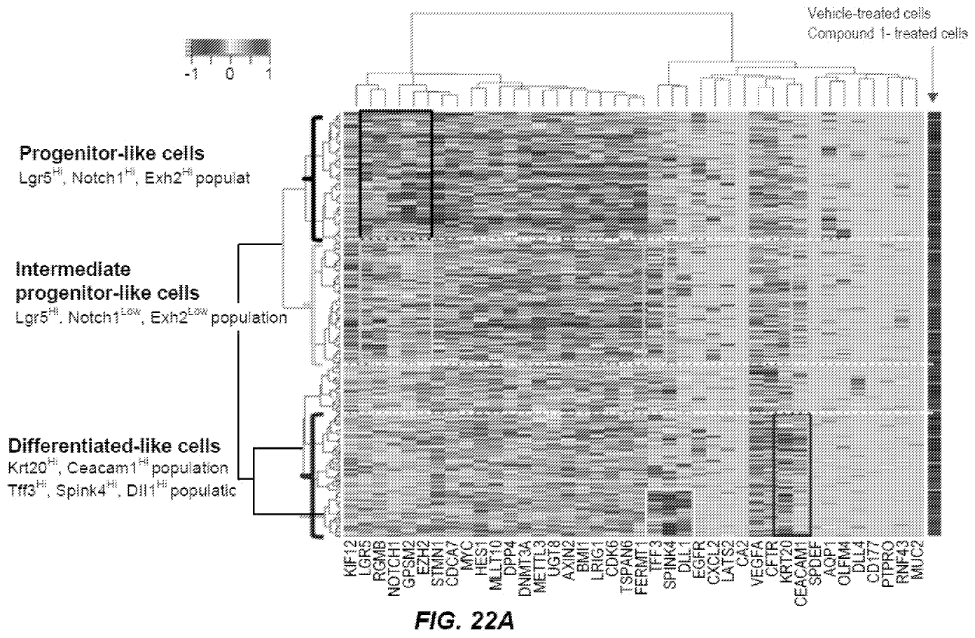

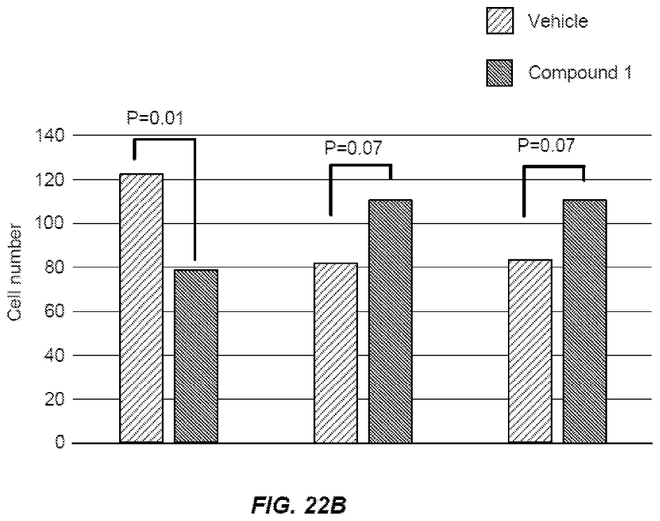

Vehicle control and Compound 1-treated samples were characterized by single cell transcriptional profiling with a gene-set designed to discriminate immature colon progenitor cells and more mature cell-types (Dalerba, 2011). Compound 1-treated tumors demonstrated a shift in cellularity upon KDM4C inhibition (FIG. 22, panel A). Using hierarchical clustering analysis, three clusters based on expression of a subset of immature and mature cell markers were identified. Progenitor-like cell clusters were identified by the presence of high expression of immature cell markers (Lgr5, Notch1 and Ezh2) and low expression for mature cell markers (Krt20, Ceacam1, and Spink4). Intermediate progenitor-like cells were identified by the presence of high expression of Lgr5 but low expression of other immature cell markers such as Notch1, Ezh2. This cluster also expressed several markers of mature cells at higher levels than the progenitor-like cells but expressed cell cycle markers at lower levels suggesting that these cells were less proliferative than the progenitor-like cells (data not shown). Finally, a cluster of differentiated-like cells expressing low levels of immature cells markers (Lgr5, Notch1, Ezh2) and high levels of mature cells markers (Krt20/Ceacam1 and Tff3/Dll1/Spink4) were identified. Compound 1-treated tumors had a significant reduction in the proportion of progenitor-like cells (p=0.01) and an increase in number of both intermediate-like and differentiated-like cells although the increase was not significant (p=0.07; FIG. 22, panel B).

### Example 8: Effect of Compound 1 on Hematopoietic Progenitor Cells

Compound 1 was evaluated for its effects on proliferation of erythroid and myeloid hematopoietic progenitor cells derived from human bone marrow at STEMCELL Technologies Inc. (Vancouver, BC, Canada).

Cells were thawed rapidly at 37° C. into DNAse I and then diluted in 10 mL of Iscove's Modified Dulbecco's medium containing 2% fetal bovine serum (IMDM+2% FBS) and washed by centrifugation (1200 rpm for 10 minutes at room temperature). The supernatant was discarded and the cell pellet resuspended in a known volume of IMDM+2% FBS. A nucleated cell count (3% glacial acetic acid) and viability assessment (trypan blue exclusive test) were performed.

Compound 1 and positive control (panobinostat) were diluted in DMSO and tested at concentrations ranging from 0.002 μM to 5 μM in final media. These dilutions provided the appropriate final test concentrations required for the test and control articles (5, 1.67, 0.56, 0.19, 0.06, 0.02, 0.07, and 0.002 μM).

Test and control articles were incubated with human bone marrow hematopoietic progenitor cells. After 14 days, cell colonies were assessed.

To calculate the concentration of 50% and 90% inhibition of colony growth (IC₅₀ and IC₉₀) for each test article, a dose response curve was generated plotting the log of the test article concentration versus the percentage of control colony growth using GraphPad Prism. To generate a curve fitting these data points, a log (inhibitor) vs. response-variable slope (four parameters) equation [Y=Bottom+(Top−Bottom)/(1+10\hat{ }((Log IC₅₀−X)*HillSlope))] was used. The IC₅₀ and IC₉₀ values for both test and control articles were derived from an interpolation of the curve fit. In cases where the IC_{50/90} value reported by GraphPad Prism is a value beyond the range of concentrations that were actually tested, if this value is more than 4 times the highest concentration tested, or where GraphPad Prism cannot calculate an IC_{50/90} value, notations of >5 μM (for test article compound 1) are used.

Compound 1 had no significant effect compared to 0.1% DMSO on the colony formation of progenitor cells from normal human bone marrow (p>0.01). As shown below in Table 12, IC₅₀ values for inhibition of erythroid and myeloid colony formation were greater than 5 μM. Statistical analysis photographs taken of representative erythroid and myeloid derived colonies found that their morphology (size and density) was slightly compromised at compound 1 concentrations≥1.67 μM. Panobinostat, the positive control, inhibited erythroid and myeloid colony formation with IC₅₀ values less than 0.01 μM.

TABLE 12

| | Erythroid | | Myeloid | |
|---|---|---|---|---|
| Test Material | IC₅₀ (μg/mL) | IC₉₀ (μg/mL) | IC₅₀ (μg/mL) | IC₉₀ (μg/mL) |
| Compound 1 | >5 | >5 | >5 | >5 |
| Panobinostat | 0.0064 | 0.0075 | 0.0074 | 0.0081 |

What is claimed is:

1. A method of treating a cancer in a cancer patient in need thereof, comprising administering to the patient a therapeutically effective amount of a compound, or a pharmaceutically acceptable salt or a solvate thereof, wherein the compound having the structure:

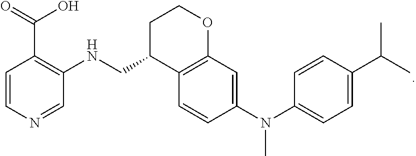 [SMILES: CC(C)c1ccc(N(C)c2ccc3c(c2)OCC[C@H]3CNc2cnccc2C(=O)O)cc1]

2. A method of reducing tumorigenic cell population in cancer a patient in need thereof, comprising administering to the patient therapeutically effective amount of a compound, or a pharmaceutically acceptable salt or a solvate thereof, wherein the compound having the structure:

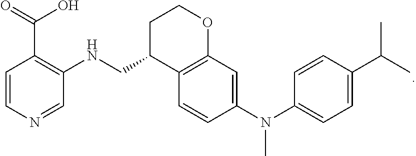 [SMILES: CC(C)c1ccc(N(C)c2ccc3c(c2)OCC[C@H]3CNc2cnccc2C(=O)O)cc1]

3. A method of reducing tumor initiating cell frequency in a cancer patient in need thereof, comprising administering to the patient therapeutically effective amount of a compound, or a pharmaceutically acceptable salt or a solvate thereof, wherein the compound having the structure:

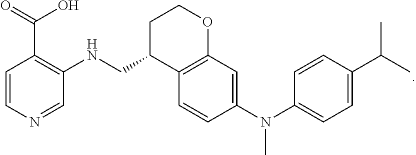 [SMILES: CC(C)c1ccc(N(C)c2ccc3c(c2)OCC[C@H]3CNc2cnccc2C(=O)O)cc1]

4. A method of inhibiting cancer stem cells in a cancer patient in need thereof, comprising administering to the patient therapeutically effective amount of a compound, or a pharmaceutically acceptable salt or a solvate thereof, wherein the compound having the structure:

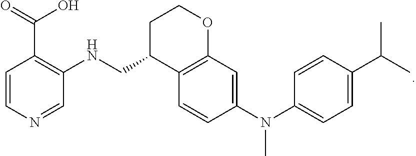 [SMILES: CC(C)c1ccc(N(C)c2ccc3c(c2)OCC[C@H]3CNc2cnccc2C(=O)O)cc1]

5. The method of any one of claims 1-4, wherein the cancer patient has been diagnosed with a cancer selected from the group consisting of colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, and T-cell acute lymphoblastic leukemia.

6. The method of any one of claims 1-4, wherein the cancer patient has been diagnosed with colorectal cancer.

7. The method of any one of claims 1-4, wherein the cancer patient has been diagnosed with esophageal cancer.

8. The method of any one of claims 1-4, wherein the cancer patient has been diagnosed with triple negative breast cancer.

9. The method of any one of claims 1-4, wherein the cancer patient has been diagnosed with gastric cancer.

10. The method of any one of claims 1-4, wherein the cancer patient has been diagnosed with lymphoma.

11. The method of any one of claims 1-4, wherein the cancer patient has been diagnosed with gastric adenocarcinoma.

12. The method of any one of claims 1-4, wherein the cancer patient has been diagnosed with diffuse large B-cell non-Hodgkin's lymphoma.

13. The method of any one of claims 1-4, wherein the cancer patient has been diagnosed with acute T-cell leukemia.

14. The method of any one of claims 1-4, wherein the cancer patient has been diagnosed with esophageal squamous cell carcinoma.

15. The method of any one of claims 1-4, wherein the cancer patient has been diagnosed with multiple myeloma.

16. The method of any one of claims 1-4, wherein the cancer patient has been diagnosed with acute myeloid leukemia.

17. The method of any one of claims 1-4, wherein the cancer patient has been diagnosed with colorectal adenocarcinoma.

18. The method of any one of claims 1-4, wherein the cancer patient has been diagnosed with colorectal carcinoma.

19. The method of any one of claims 1-4, wherein the cancer patient has been diagnosed with pancreatic cancer.

20. The method of any one of claims 1-4, wherein the cancer patient has been diagnosed with pancreatic carcinoma.

21. The method of any one of claims 1-4, wherein the cancer patient has been diagnosed with breast carcinoma.

22. The method of any one of claims 1-4, wherein the cancer patient has been diagnosed with T-cell acute lymphoblastic leukemia.

23. The method of any one of claims 1-4, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma.

24. The method of any one of claims 1-4, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia.

25. The method of any one of claims 1-4, wherein the cancer patient has been diagnosed with a cancer selected from intestinal cancer, intestinal adenocarcinoma, squamous cell carcinoma of the upper digestive tract.

26. The method of any one of claims 1-4, wherein the cancer patient has been diagnosed with a cancer selected from stomach cancer, stomach signet ring adenocarcinoma, adenocarcinoma of the stomach, or adenosquamous carcinoma.

27. The method of any one of claims 1-4, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

28. The method of any one of claims 1-4, wherein the cancer patient has been diagnosed with skin cancer.

29. The method of any one of claims 1-4, wherein the cancer patient has been diagnosed with a cancer selected from thyroid cancer, or thyroid follicular carcinoma.

30. The method of any one of claims 1-4, wherein the cancer patient has been diagnosed with a cancer selected from breast cancer, or breast ductal carcinoma.

31. The method of any one of claims 1-4, wherein the cancer patient has been diagnosed with a cancer selected from liver cancer, or hepatocellular carcinoma.

32. The method of any one of claims 1-4, wherein the cancer patient has been diagnosed with a cancer selected from a CNS cancer, astrocytoma grade IV, or gliosarcoma.

33. The method of any one of claims 1-4, wherein the cancer patient has been diagnosed with bone cancer.

34. The method of any one of claims 1-4, wherein the cancer patient has been diagnosed with a cancer selected from kidney cancer, clear cell renal cell carcinoma, renal cell carcinoma, urinary tract cancer, or urinary tract transitional cell carcinoma.

35. The method of any one of claims 1-34, wherein the cancer is relapsed after prior therapy, refractory to prior therapy, or acquired resistance to prior therapy.

36. The method of any one of claims 2, and 5-35, wherein the tumorigenic cell population is reduced from about 1.5-fold to about 100-fold.

37. The method of claim 36, wherein the tumorigenic cell population is reduced about 1.5-fold, about 2.0-fold, about 2.5-fold, about 3.0-fold, about 3.5-fold, about 4.0-fold, about 4.5-fold, about 5.0-fold, about 6.0-fold, about 7.0-fold, about 8.0-fold, about 9.0-fold, about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, or about 100-fold.

38. The method of claim 36, wherein the tumorigenic cell population is reduced about 1.5-fold, about 2.0-fold, about 2.5-fold, about 3.0-fold, about 3.5-fold, about 4.0-fold, about 4.5-fold, or about 5.0-fold.

39. The method of any one of claims 3, and 5-35, wherein the tumor initiating cell frequency is reduced from about 1.5-fold to about 100-fold.

40. The method of claim 39, wherein the tumor initiating cell frequency is reduced about 1.5-fold, about 2.0-fold, about 2.5-fold, about 3.0-fold, about 3.5-fold, about 4.0-fold, about 4.5-fold, about 5.0-fold, about 6.0-fold, about 7.0-fold, about 8.0-fold, about 9.0-fold, about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, or about 100-fold.

41. The method of claim 39, wherein the tumor initiating cell frequency is reduced about 1.5-fold, about 2.0-fold, about 2.5-fold, about 3.0-fold, about 3.5-fold, about 4.0-fold, about 4.5-fold, about 5.0-fold, about 6.0-fold, about 7.0-fold, about 8.0-fold, about 9.0-fold, or about 10-fold.

42. The method of any one of claims 3, and 5-35, wherein the tumor initiating cell frequency is reduced to a frequency of about 1-in-100 cells to about 1-in-10,000 cells.

43. The method of claim 42, wherein the tumor initiating cell frequency is reduced to a frequency of about 1-in-100 cells, about 1-in-200 cells, about 1-in-300 cells, about 1-in-400 cells, about 1-in-500 cells, about 1-in-600 cells, about 1-in-700 cells, about 1-in-800 cells, about 1-in-900 cells, about 1-in-1,000 cells, about 1-in-1,200 cells, about 1-in-1,400 cells, about 1-in-1,600 cells, about 1-in-1,800 cells, about 1-in-2,000 cells, about 1-in-3,000 cells, about 1-in-4,000 cells, about 1-in-5,000 cells, about 1-in-6,000 cells, about 1-in-7,000 cells, about 1-in-8,000 cells, about 1-in-9,000 cells, or to about 1-in-10,000 cells.

44. The method of claim 42, wherein the tumor initiating cell frequency is reduced to a frequency of about 1-in-200 cells, about 1-in-300 cells, about 1-in-400 cells, about 1-in-500 cells, about 1-in-600 cells, about 1-in-700 cells, about 1-in-800 cells, about 1-in-900 cells, about 1-in-1,000 cells, or about 1-in-1,200 cells.

45. A method of treating a cancer in a cancer patient in need thereof, comprising:
(a) determining high microsatellite instability (MSI-H) status of the cancer patient, and
(b) administering therapeutically effective amount of a compound or a pharmaceutically acceptable salt or a solvate thereof to the cancer patient if the cancer patient is determined to be a MSI-H cancer patient, wherein the compound having the structure:

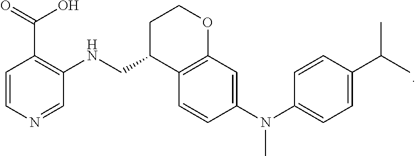 [SMILES: CC(C)c1ccc(N(C)c2ccc3c(c2)OCC[C@H]3CNc2cnccc2C(=O)O)cc1]

46. A method of treating cancer, comprising:
(a) obtaining a sample from a patient having the cancer;
(b) determining the level of a biomarker in the sample;
(c) diagnosing the patient as being likely to be responsive to a treatment compound if the level of the biomarker in the sample is different from a reference level of the biomarker;
and (d) administering a therapeutically effective amount of the treatment compound to the patient; and
wherein the treatment compound is Compound 1, or a pharmaceutically acceptable salt or a solvate thereof; and
the biomarker is PNUTS.

47. The method of claim 33 or 34, wherein the cancer patient has been diagnosed with a cancer selected from the group consisting of colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, and T-cell acute lymphoblastic leukemia.

48. The method of claim 33 or 34, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma.

49. The method of claim 33 or 34, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia.

50. The method of claim 33 or 34, wherein the cancer patient has been diagnosed with a cancer selected from intestinal cancer, intestinal adenocarcinoma, squamous cell carcinoma of the upper digestive tract.

51. The method of claim 33 or 34, wherein the cancer patient has been diagnosed with a cancer selected from stomach cancer, stomach signet ring adenocarcinoma, adenocarcinoma of the stomach, or adenosquamous carcinoma.

52. The method of claim 33 or 34, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

53. The method of claim 33 or 34, wherein the cancer patient has been diagnosed with skin cancer.

54. The method of claim 33 or 34, wherein the cancer patient has been diagnosed with a cancer selected from thyroid cancer, or thyroid follicular carcinoma.

55. The method of claim 33 or 34, wherein the cancer patient has been diagnosed with a cancer selected from breast cancer, or breast ductal carcinoma.

56. The method of claim 33 or 34, wherein the cancer patient has been diagnosed with a cancer selected from liver cancer, or hepatocellular carcinoma.

57. The method of claim 33 or 34, wherein the cancer patient has been diagnosed with a cancer selected from a CNS cancer, astrocytoma grade IV, or gliosarcoma.

58. The method of claim 33 or 34, wherein the cancer patient has been diagnosed with bone cancer.

59. The method of claim 33 or 34, wherein the cancer patient has been diagnosed with a cancer selected from kidney cancer, clear cell renal cell carcinoma, renal cell carcinoma, urinary tract cancer, or urinary tract transitional cell carcinoma.

60. The method of claim 33 or 34, wherein the cancer is relapsed after prior therapy, refractory to prior therapy, or acquired resistance to prior therapy.
